(19) United States
(12) Patent Application Publication
(10) Pub. No.: US 2015/0208621 A1
(43) Pub. Date: Jul. 30, 2015

(54) Transgenic Animal

(71) Applicant: Foundation for Research & Technology Hellas-FORTH, Heraklion, OT (GR)

(72) Inventor: George Mavrothalassitis, Heraklion (GR)

(21) Appl. No.: 14/420,638

(22) PCT Filed: Aug. 3, 2013
(86) PCT No.: PCT/EP2013/066341

§ 371 Date: Feb. 9, 2015

(30) Foreign Application Priority Data
Aug. 10, 2012 (GB) 1214337.6

(51) Int. Cl. A01K 67/027; C12N 15/85

(52) U.S. Cl. CPC A01K 67/0276; C12N 15/8509; A01K 2267/03

(57) ABSTRACT

The present invention concerns a non-human transgenic animal comprising a modification in its genome which results in the animal exhibiting a level of Erf expression and/or ERF activity which is reduced compared to a wildtype animal, wherein said animal has a defect in ossification. Further provided is a non-human transgenic animal comprising an insertion of a promoter-marker gene cassette in one or both Erf alleles wherein the cassette is present in an intron of Erf and wherein said animal exhibits an expression level of Erf and/or ERF activity which is reduced compared to a wildtype animal. Uses of these animals and of a non-human transgenic animal comprising a modification in its genome which results in the animal exhibiting a level of expression of Erf and/or ERF activity which is reduced compared to a wildtype animal for producing animals with ossification defects, for identifying treatments and as disease models are encompassed.

The present invention relates to an animal model for defects in ossification, and in particular for craniosynostosis. This invention is based on the novel and surprising finding that mutations in the gene encoding the transcription factor ERF are involved in the pathogenesis of craniosynostosis and in particular that reduced levels of ERF expression and/or activity may lead to the development of defects in ossification. Accordingly an animal model in which ERF expression and/or activity is reduced may be used for the study of such defects and to study or identify therapeutic agents for potential use in the treatment of such conditions.

ERF is a ubiquitously expressed inhibitory transcription factor that is part of the ets family of transcription factors. Whilst most of the ets family are transcriptional activators, ERF was the first mammalian ets family member to be identified as a transcriptional repressor. Particularly, ERF has been shown to be responsible for the transcriptional regulation of the ETS2 gene (U.S. Pat. No. 5,856,125).

ERF comprises several functionally characterised motifs, including an N-terminal DNA binding motif (ets), a central ERK1/2 interaction motif and a C-terminal repressor domain. ERF has been reported to interact with and to be phosphorylated by ERK 1/2, this phosphorylation diminishing the repressor activity of ERF by causing ERF export from the nucleus. It has further been reported that activation of ras/MAPK inhibits ERF.

In addition to acting as a transcriptional repressor, it has been proposed that ERF may have tumour suppressor activity and particularly may be able to suppress ets-dependent tumourigenecity. It has therefore been suggested that ERF expression may be beneficial in reducing such tumourigenecity. As it is possible to transfer the ERF repressor activity to other transcription factors by producing chimeric molecules comprising the ERF repressor domain attached to the transcription factor binding site, it is also possible to use ERF in this way to reduce tumourigenecity associated with the aberrant activation of such transcription factors. Thus, ERF has been investigated previously in view of its use as a tumour suppressor.

ERF has further been identified as having a role in extraembryonic ectoderm differentiation. In this regard, homozygous deletion of Erf in mice leads to a block of chorionic cell differentiation before chorioallantoic attachment, persisting chorion layer, failure of chorioallantoic attachment and absence of labyrinth, which in turn result in embryo death by 10.5 dpc. Heterozygous mice did not show any obvious phenotypic abnormality (Papadaki et al, Mol. Cell Biol., 27, 5201-5213, 2007).

Craniosynostosis is a condition which affects approximately 1 in 2500 children, in which at least one cranial suture is prematurely fused by ossification. This premature fusion changes the growth pattern of the skull, which typically results in visible abnormalities of head shape and/or facial features (cranio-facial defects), and which, depending on the severity of the condition, can lead to other complications which result from insufficient space for the brain to grow, leading to increased intra-cranial pressure (e.g. visual impairment, sleeping impairment, eating difficulties or an impairment of mental development and reduction in IQ).

The disease has many different presentations and causes, not all of which are known, and may involve the early fusing of a single suture (e.g. the sagittal, coronal, metopic or lambdoid suture), or may involve the early fusing of multiple sutures. Craniosynostosis may further be non-syndromic (i.e. an isolated condition, seen in isolated patient cases, not linked to any particular condition) or syndromic, where it is part of a a clinical syndrome an other clinical disease signs are present. Non-syndromic cases with single suture fusions can often be corrected by surgery, but cases with multiple suture fusions may be more difficult to handle.

Several genetic mutations have been identified in patients with craniosynostosis. Heterozygous mutations in FGFR2 (fibroblast growth factor receptor 2) have been shown to cause Crouzon, Apert and Pfeiffer syndromes and heterozygous mutations in TWIST1 (twist homolog 1) has been shown to cause Saethre-Chotzen syndrome. Further, mutations in EFNB1 (ephrin B1) may cause craniofrontonasal syndrome. The identification of mutations is important for the diagnosis and prognosis of patients with the condition, to ensure prompt and appropriate treatment for the disease. In this regard, one of the most important mutations that has been identified to date is a heterozygous P250R mutation in FGFR3 which has been shown to be associated with coronal synostosis, a condition that may have non-specific clinical features and where craniosynostosis may not always occur. However, the genetic mutations so far associated with craniosynostosis are only present in approximately 25% of patients. There may therefore be several other genetic causes (or other causes) of craniosynostosis which have not been identified.

The present inventors have now surprisingly discovered that mutations in the Erf gene may be associated with the development of craniosynostosis, and on this basis have identified a new disorder, termed ERF-related craniosynostosis. This newly-identified condition thus represents a sub-group of the general disorder craniosynostosis, which as indicated above may have a number of causes. ERF-related craniosynostosis was identified in 5/402 (1.2%) of all patients requiring surgery for craniosynostosis in the cohort of patients studied. Thus, mutations were identified in the Erf gene in a number of patients with craniosynostosis and genetic analysis has established the Erf mutations to be the cause of the craniosynostosis in the affected families. Further work has shown that reduced dosage of ERF, e.g. a reduction of the expression level of the Erf gene may cause a craniosynostosis phenotype. However, not all the observed mutations which are correlated with the disease phenotype appear to be associated with reduced expression and thus other mutations may affect ERF function (or activity) rather than expression (for example mutations in the DNA-binding domain may result in loss of repressor activity). The data appear to indicate that the predominant pathological mechanism is heterozygous loss of function (haploinsufficiency).

As discussed above, from a clinical viewpoint, ERF has previously been associated only with tumour suppressing activity and a role in ossification defects, and particularly craniosynostosis, has not been previously been suggested. The present inventors analysed DNA from two brothers, both with craniosynostosis (one brother had metopic, sagittal and left coronal sutures affected and the other brother had multisuture synostosis). Their mother exhibited exorbitism and midface hypoplasia, but not craniosynostosis. A mutation in the Erf gene was detected in the brothers (547C to T, resulting in an amino acid alteration at position 183 from arginine to a stop) and was shown to segregate from the maternal grandmother to the two affected children. This finding led to further genetic studies is which the Erf gene was sequenced in unrelated craniosynostosis patients and other occurrences of mutations were identified in some patients and their families (but never in normal controls). The mutations identified are diverse e.g. missense in the initiation codon or in critical residues in the DNA-binding ets domain, splice site mutation, nonsense changes and frameshift mutations and cause a variety of phenotypes in affected patients. On this basis, it is proposed according to the present invention that reduction of ERF expression and/or activity may lead to premature cranial suture fusion and/or other defects of ossification. This has led the present inventors to develop the animal model of the present invention.

As discussed above, a role for ERF was not previously suspected in either the cranial sutures or in osteogenesis more generally. In addition to the surprising identification of Erf mutations in craniosynostosis in human patients, and the preliminary work suggesting that reduced Erf gene expression or reduced ERF activity leads to the phenotypic manifestation of the ossification defects, a mouse model has been developed in which the effects of reduced ERF expression and/or activity can be studied.

In particular, the inventors have now further shown that a transgenic mouse having a reduced expression level of Erf displays a phenotype showing features of craniosynostosis (i.e. craniosynostosis phenotype). This was unexpected in view of the previous finding that a mouse with a heterozygous Erf wildtype/null genotype presented a normal phenotype.

In a first aspect, the present invention provides a non-human transgenic animal comprising a modification in its genome which results in the animal exhibiting a level of Erf expression and/or ERF activity which is reduced compared to a wildtype animal, wherein said animal has a defect in ossification.

In particular the animal displays or exhibits a phenotype which comprises a defect in ossification (i.e. the animal displays or exhibits a phenotypic defect of ossification).

The term “wild-type” as used herein may denote that the animal does not contain the modification which is introduced into the transgenic animal to cause the reduction in ERF expression and/or activity. Thus according to the present invention the transgenic animal may display a reduced level of ERF expression and/or activity as compared with (or relative to) an animal which does not contain the modification. It is not precluded that either the transgenic animal of the invention or the reference animal used for the comparison contains other modifications which are not related to ERF expression and/or activity. e.g. other modifications to the genome which are not present in a native animal.

Hence, the present invention lies primarily in the production of a transgenic non-human animal which displays a defect in ossification, and preferably has craniosynostosis. Such animals can be used as disease models for ossification defects, and in particular for craniosynostosis, as discussed below in detail, and may be used to identify various possible disease treatments and to study the disease. However, as will be discussed further below, in certain aspects the invention also extends to transgenic animals and their use, which have reduced ERF expression and/or activity, but which do not have defects of ossification, or indeed which do not display an altered phenotype or any disease phenotype e.g. which have a normal phenotype.

The term “transgenic animal” as used herein means an animal into which a genetic modification has been introduced by a genetic engineering procedure and in particular an animal into which has been introduced an exogenous nucleic acid. That is the animal contains or comprises a nucleic acid molecule or a nucleotide sequence which is not normally present in the animal or which is present in the animal in a location or at a site in which it is not normally present. The sequence may thus be a foreign or a non-native sequence. In particular the nucleotide sequence may be a recombinant nucleic acid molecule or construct. Included are both progenitor and progeny animals, “progeny” animals including animals which are descended from the progenitor as a result of sexual reproduction or cloning and which have inherited genetic material from the progenitor. Thus, the progeny animals comprise the genetic modification introduced into the parent. A transgenic animal may be developed from embryonic cells into which the genetic modification (e.g. exogenous nucleic acid molecule or nucleotide sequence) has been directly introduced or from the progeny of such cells. The exogenous nucleic acid is introduced artificially into the animal (e.g. into a founder animal) and thus insofar as such animals are concerned, the introduction of a nucleic acid into the transgenic animal through normal reproductive processes (such as breeding) is excluded, as are naturally or spontaneously occurring mutations. However, animals that have been produced by transfer of an exogenous nucleic acid through breeding of the animal comprising the nucleic acid (into whom the nucleic acid was artificially introduced) and which are accordingly “progeny” animals are expressly included.

As discussed further below, the exogenous nucleic acid may be integrated into the genome of the animal or it may be present in an non-integrated form, e.g. as an autonomously-replicating unit, for example an artificial chromosome which does not integrate into the genome but which is maintained and inherited substantially stably in the animal.

As indicated above, the non-human transgenic animals referred to herein have a reduced level of Erf expression and/or ERF activity. In preferred or particular embodiments the animal exhibits a level of Erf expression and/or ERF activity which is less than 50% of the Erf expression level and/or ERF activity of a wildtype animal. Such a wild-type animal may be an animal of the same species or the same strain without the genetic modification which is introduced in the transgenic animal and which is responsible for the reduction in Erf expression and/or ERF activity. Thus the wild-type animal may be seen as a corresponding animal which does not have the genetic modification. Thus the wildtype animal may be a normal animal without the transgenic genotype and which expresses a normal amount of Erf and/or whose ERF has normal activity. Although Erf has a ubiquitous expression pattern, it is preferred in the present invention that Erf expression levels and/or ERF activity comparisons between the non-human transgenic animals discussed herein and wildtype or reference animals are made using samples derived from the same tissue types or bodily fluids e.g. if a blood sample is used to determine the level of Erf expression of a transgenic animal then a blood sample is also used to determine the level of Erf expression of a wildtype animal. In particular embodiments, the transgenic animal has less than 50% Erf expression compared to a wildtype non-human animal and/or has less than 50% ERF activity compared to a wildtype non-human animal.

In the first aspect of the invention indicated above, the reduction in the level of expression of Erf and/or the reduction in the activity of ERF is sufficient to result in a disease phenotype i.e. in ossification defects such as craniosynostosis, although as mentioned above and discussed further below, the present invention may in certain embodiments extend to non-human transgenic animals which have a reduced level of expression of Erf and/or activity of ERF but which do not have a ossification disease phenotype. Such animals may be used to generate animals of the invention and/or may have utility in identifying other non-documented phenotypes/conditions associated with carrying a genetic modification which affects Erf expression/ERF activity.

Work underlying the present invention has shown that expression of the disease phenotype is sensitive to ERF dosage. Accordingly, it is believed that there is a threshold level of activity or expression, below which the disease phenotype is manifested. This threshold may vary in different animals. Thus, in humans haploinsufficiency (i.e. one mutant allele) may lead to disease. However, in mice ERF^{+/-} heterozygotes have been shown to be phenotypically normal. Accordingly in the transgenic animals of the present invention, the level of ERF expression and/or activity is in certain embodiments reduced to a level which results in the disease phenotype. It may not in certain such embodiments be sufficient to knock out one copy of the Erf gene for example. As discussed further below, this may in certain embodiments e.g. depending on the nature of the modification, and/or in certain species of animal, require that compound heterozygotes are made, comprising a combination of different genetic modifications.

In a preferred aspect of the invention, the level of expression of Erf and/or ERF activity is reduced to less than 50% of a wildtype non-human animal, wherein the reduction in Erf expression and/or ERF activity is sufficient to result in a defect in ossification. Particularly, in this and also in other aspects and embodiments of the invention as set out herein, the non-human transgenic animal may exhibit a reduction in Erf expression and/or ERF activity of at least 50, 51, 52, 53, 54, 55, 60, 65, 70, 75, 80, 85, 90 or 95%. Thus, the animal may exhibit less than 50% or no more than, or up to, 49, 47, 45, 42, 40, 37, 35, 32 or 30% of the level of Erf expression and/or ERF activity of a wild-type animal, or no more than or up to a percentage indicated by any integer between 30 and 49. However, the non-human transgenic animal should exhibit some Erf expression and/or have some ERF activity e.g. at least 1, 2, 3, 4, 5, 6, 7, 8, 10, 11, 12, 13, 14, 15, 16, 17, 18, 19 or 20%, in order to prevent embryonic death.

Thus, alternatively defined, Erf expression and/or ERF activity in a non-human transgenic animal may be from 5-49% of the Erf expression and/or ERF activity of a wildtype animal, or any range between any of the integers therebetween e.g. between 5-45, 5-42, 5-40, 5-37, 5-35, 5-32, 5-30, 10-49, 10-47, 10-45, 10-42, 10-40, 10-37, 10-35, 10-32, 10-30, 12-49, 12-47, 12-45, 12-42, 12-40, 12-37, 12-35, 12-32, 12-30; 15-49, 15-47, 15-45, 15-42, 15-40, 15-37, 15-35, 15-32, 15-30, 20-49, 20-47, 20-45, 20-42, 20-40, 20-37, 20-35, 20-32, 20-30, 25-49, 25-47, 25-45, 25-42, 25-40, 25-37, 25-35, 25-32, 25-30, 30-49, 30-47, 30-45, 30-42, 30-40, 35-45, 35-40 or 40-45% etc. of the Erf expression level and/or ERF activity of a wildtype animal.

Erf expression levels and/or ERF activity may be measured using a sample of tissue or fluid obtained from the non-human transgenic animal. Such expression/activity levels may be compared to those in a wildtype animal (preferably those found in the same tissue/fluid). The wildtype expression levels/activity levels may have already been pre-determined or may be identified at the same time as the measurements of expression/activity levels for the non-human transgenic animal. The sample may be any sample obtained from the transgenic animal, in view of the ubiquitous expression pattern of Erf but particularly may be a blood sample, a bone marrow biopsy, a bone sample, or a tail snip sample, particularly if the non-human transgenic animal is a mouse.

A reduction in the level of Erf expression may be determined by measuring Erf mRNA present in a sample and/or the amount of ERF protein produced. Erf mRNA levels may be determined using any techniques which are well known in the art e.g. Northern blotting or microarray technology. Further, real time PCR can be used to determine mRNA levels, where total RNA can be extracted from a cell and reverse transcribed. Real time PCR can then be carried out on the reverse transcribed sample and the mRNA expression level determined. ERF protein levels can be determined using many well known techniques. Antibodies which bind to ERF can be used in any of the well known immunoassay techniques which are widespread in the art e.g. sandwich assays, competitive assays, immunometric assays etc. Other assay formats may also be used e.g. assays based on flow cytometry. Western blotting may be used where proteins are resolved by SDS-PAGE and transferred to nitrocellulose membranes. ERF can then be detected and its level measured by using an anti-ERF antibody. Such anti-ERF antibodies are available from several well known manufacturers and include ERF antibodies C-20, H-68 and 33-L (from Santa Cruz Biotechnology, Inc, codes sc15435, sc292179 and sc130372, respectively), ERF antibody 3F11 (from AbD Serotec, code MCA40207) and a polyclonal ERF antibody from Lifespan Biosciences (code LS-C117642). The C-20 and H-68 antibodies may be used to identify ERF from mouse, rat, human, equine, canine, bovine and porcine sources, the 33-L antibody may be used to identify ERF from mouse and human sources, the 3F11 antibody may detect murine ERF and the polyclonal Lifespan Biosciences antibody can be used to detect rabbit ERF. Further, antibodies for detecting ERF may be made de novo if desired using techniques which are well known in the art.

The antibody may be labelled and may be detected and/or measured by means of the label. Labelling may be by any convenient means and a wide variety of labels and labelling techniques for antibodies are well known in the art. Such labels may include for example, fluorochromes, radioisotopes, coloured dyes, quantum dots, or other chromogenic agents, enzymes, colloidal metals, chemi and bio-luminescent compounds. The labels may be directly detectable or signal giving such as those listed above, or they may be labels which take part in a signal giving or detectable reaction, for example by binding to another molecule e.g. they may be indirectly detected. Thus a label may be a small molecule such as a hapten or a tag e.g. biotin which may be bound by a binding partner therefor e.g. streptavidin/avidin for biotin.

Further, ERF protein levels may be determined by immunohistochemistry.

The murine Erf mRNA transcribed from the murine Erf gene has a nucleic acid sequence as set forth in SEQ ID NO. 1. The sequence of murine ERF protein is as set forth in SEQ ID NO. 2. A genomic sequence showing the nucleotide sequence of the mouse Erf gene along with some flanking sequence is shown in SEQ ID NO. 7. The full length of the sequence shown in SEQ ID NO. 7 is 1-10199 nucleotides and the mouse Erf gene sequence lies at nucleotides 1001-9199 of SEQ ID. NO. 7 (mRNA is join (1001 . . . 1180, 5437 . . . 5671, 6010 . . . 6125, 6228 . . . 9199) and the CDS is join (1159 . . . 1180, 5437 . . . 5671, 6010 . . . 6125, 6228 . . . 7510)). Both the mouse Erf gene and ERF protein sequences and corresponding (orthologous) sequences from other animal species are freely available and may be obtained from publically available databases (e.g. Genbank). Thus, for example, the measurement of mRNA expressed in a transgenic animal discussed herein or the measurement of the ERF protein in the transgenic animal, refers to the measurement of an mRNA sequence of SEQ ID NO. 1 or mRNA transcribed from an orthologous gene thereto or to the measurement of the levels of a protein of SEQ ID NO. 2 or an orthologous protein thereto. It will be appreciated by a skilled person that where the transgenic animal is a mouse, murine Erf mRNA may be measured and/or murine ERF protein levels may be measured to determine whether the mouse exhibits a reduced level of Erf expression. Alternatively, if the transgenic animal is a rabbit, rat, dog, cat etc, then Erf mRNA or protein from each of those animals may be measured. Thus, the Erf mRNA/protein sequence which is measured will be that which is particular to the transgenic animal species which has been made. Further, as the Erf gene in the transgenic animal may have been modified (i.e. to result in an animal which exhibits a reduced level of expression of Erf and/or ERF activity), the measurement of Erf mRNA and/or protein levels may involve measuring a mutant form of Erf mRNA and/or protein. Particular mutations which may be made to the Erf gene and thus which may be present in any transcribed mRNA and/or translated protein are discussed in detail below but include an addition, deletion, or substitution of one or more nucleotides. For transgenic animals which contain heterozygous Erf gene mutations or which contain compound Erf gene mutations (i.e. different mutations on each Erf allele), it may be possible to determine whether Erf expression is reduced by measuring either only the expression of the wildtype allele or the expression of the mutant allele. Alternatively, expression can be measured for both alleles.

As discussed above, ERF activity may be alternatively or additionally reduced in the non-human transgenic animal referred to herein. ERF activity may be determined by assessing its ability to repress the ETS2 gene transcription or by assessing its ability to repress any of its other targets. Thus, ERF activity can be measured by measuring the levels of a downstream target of ERF e.g. measuring the mRNA/protein levels of such a target using any of the methods described above for Erf. Particularly, where ERF is known to repress a particular target gene e.g. ETS2, an increase in the expression of such genes may be indicative of reduced ERF activity. Thus, reduced ERF activity could be measured by determining the mRNA transcribed from a gene which is usually repressed by ERF (or by measuring the level of translated protein from that gene). The exact type of ERF activity which is reduced by the genotype modification may depend on exactly what genotype modification is made and where it is made.

The transgenic non-human animal described herein comprises a genetic modification which results in the animal exhibiting a reduced level of Erf expression and/or ERF activity compared to a wildtype animal. Any genetic modifications which have the desired effect on Erf expression levels and/or ERF activity are encompassed. Thus, the genetic modification may be made directly to any part of the Erf gene e.g. to its promoter, intron or exon sequences, or may be made to another gene whose encoded protein either directly or indirectly regulates Erf gene expression or interacts directly or indirectly with ERF protein and therefore affects its activity. Therefore modifications may be made to any part of any other gene which would have the result of reducing Erf expression and/or activity e.g. to its promoter, intron or exon sequences. Alternatively, the genetic modification may encompass the insertion of a transgene or of an exogenous nucleic acid where the expression of the transgene or the nucleic acid sequence has the effect of reducing Erf expression levels and/or ERF activity.

In one embodiment, modifications are made to the Erf gene to produce a transgenic animal as described herein. Thus, as indicated above, any part of the Erf gene may be mutated or modified, including the promoter region, the N-terminal DNA binding motif (ets), the central ERK1/2 interaction motif and/or the C-terminal repressor domain. The modification can be made to coding or non-coding sequences. Thus the sequence of the Erf gene may be modified such as to result in inactivation of the gene or the encoded gene product (e.g. a null mutation) or to result in a reduction in the level of expression or in the activity of the encoded gene product. Different mutations may be introduced into different alleles, and thus although it is a requirement that the animal must express some active ERF protein, it is not precluded that an inactivating mutation (e.g. a null mutation) is introduced into one of the alleles.

To reduce the level of expression or activity of ERF to below 50% compared to wild-type may in some embodiments require a combination of mutations in different alleles. Thus, for example one allele may be inactivated (e.g. a null mutation) and the other allele may be modified to result in reduced (rather than abrogated) expression and/or activity. This is discussed further below.

One or more modifications may be made to any part of the Erf gene or to any part of any other gene whose encoded protein either directly or indirectly regulates Erf gene expression or interacts directly or indirectly with ERF protein and therefore affects its activity e.g. to any one or more of the above described regions. Thus, for example at least 1, 2, 3, 4, 5, 6, 7, 8, 9 or 10 modifications may be made. The one or more modifications of the gene include an insertion (e.g. which may result in a frameshift mutation), a deletion (which may result in a frameshift mutation or a deletion of part or all of the gene), an inversion or a point mutation which may result in a missense or nonsense mutation. Further, one or more portions of the gene e.g. the Erf gene, may be substituted with exogenous nucleic acids.

The one or more modifications may be made to either one or both alleles of the gene e.g. the Erf gene. When a modification is made to only one of the alleles, or different modifications are made in different alleles, the modification is referred to as heterozygous and when the same modification is made to both alleles, the modification is homozygous. It is thus possible that the one or more modifications which are introduced into either or both alleles may be different. Thus each allele may comprise a different modification from the other allele. If more than one modification is being introduced into each allele, it is possible that some of the modifications may be common between the two alleles and that others may not. Any changes may be introduced into either allele as long as the result is a reduced expression level of Erf and/or of ERF activity (e.g. to less than 50% of the level in a wildtype animal and preferably an ossification defect). Further, the one or more modifications made to the one or both alleles may comprise any combination of modifications discussed above e.g. insertions, deletions, substitutions, inversions and/or point mutations. Thus, not all the modifications need to be of the same type, although they can be if desired. Hence, one or more deletions, insertions, substitutions, inversions and/or point mutations may be made to either or both alleles.

Where the Erf gene is modified, the introduced at least one mutation may result in a reduction of expression of Erf from the modified allele, a subsequent reduction in the amount of ERF protein produced and/or the production of ERF with reduced activity. Thus, in this instance, the mutation introduced may result in some expression of Erf and/or in the production of ERF with some activity e.g. between 5-50% or 5-49% of the expression/activity seen in wildtype animals. Alternatively, as noted above, the introduced at least one mutation may cause complete loss of function from that Erf allele i.e. either no Erf expression occurs or any resulting ERF protein is non-functional. Such a mutation is referred to as null. Preferably where a null mutation is introduced into one Erf allele, the other Erf allele should not contain a null mutation; any mutation in this allele should preferably allow an Erf expression level of at least 5% of that seen in a wildtype animal. Thus, where one Erf allele has a null mutation, resulting in loss of function, the other Erf allele should either be wildtype or may comprise at least one other mutation but should allow an Erf expression level and/or ERF activity of at least 5% of that seen in a wildtype animal.

As discussed further below, it is also possible for the non-human transgenic animal of the invention to comprise genetic modification of more than one gene. Therefore different genes may contain one or more modifications to either or both alleles. In this embodiment for example, a modification may be made to Erf, and to another gene whose protein product controls Erf expression and/or ERF activity. Further, the transgenic animal may comprise a genetic modification such as the insertion of exogenous nucleic acid together with the modification of one or more endogenous genes to reduce the expression level of Erf and/or the activity of ERF. Any combination of modifications can be made which results in the reduction of Erf expression and/or ERF activity.

In a particular embodiment of the present invention, one Erf allele may comprise a null mutation i.e. the allele is completely non-functional, and the other Erf allele may comprise a different modification. Such a null allele may not produce any functional ERF protein (either transcription or translation are impaired or the resulting ERF mutant protein is non-functional). Particularly, the modification introduced into the other Erf allele may further reduce Erf expression and/or ERF activity, resulting in a transgenic non-human animal which exhibits less than 50% of the Erf expression and/or ERF activity of a wildtype animal. Such a combination of modifications is particularly preferred where the transgenic animal is a rodent, e.g. a mouse.

The insertion which may be made in the Erf gene (or to any othe gene which is being modified as discussed below) may be an insertion of one or more nucleotides. Thus, the insertion may result in a frameshift mutation affecting the transcribed product and/or the translated protein. Alternatively, the insertion may be in frame, resulting in the production of a protein which is longer than that produced in a wildtype animal i.e. which comprises additional sequence within the protein sequence. Thus, the insertion may comprise at least one nucleotide base, e.g. at least 1, 2, 3, 4, 5, 6, 7, 8, 9 or 10 nucleotides or at least 20, 30, 40, 50, 100, 200, 300, 400 or 500 nucleotides. Further, the insertion may comprise the insertion of an expression cassette, e.g. comprising a promoter element, together with a further gene e.g. an exogenous gene. Such expression cassettes, preferably comprise a marker gene which allows easy detection of the presence of the insertion. In a particularly preferred aspect of the present invention, the insertion may comprise the neomycin resistance gene. Other marker genes are however used and known in the art, and any of these could be used. The promoter which may be used in an expression cassette insertion comprising an exogenous gene may be any promoter that allows expression of the gene. Particularly, the PGK promoter may be used. The insertion may comprise one or more LoxP/FRT sites. These systems are discussed in detail below, but typically allow the subsequent deletion of the nucleic acid sequence which occurs between any two such sites e.g after exposure to Cre recombinase. Alternatively, depending on the orientation of such LoxP/FRT sites, nucleic acid material may be inverted or a recombination event may occur with another gene, effectively resulting in the substitution of nucleic acid material.

A deletion refers to the deletion of at least one nucleotide from any part of a gene sequence e.g. the promoter, the exon or intron portions of the gene. Such a deletion may result in a frameshift mutation, or may allow the sequence to stay inframe, allowing the production of a protein which is shorter than that produced in a wildtype animal but which is only lacking the specific residues encoded by the nucleotides deleted in the corresponding gene. The deletion may result in the production of a truncated protein product or mRNA which is less that 95, 90, 85, 80, 75, 70, 60, 50, 40, 30 or 20% of the length of the protein or mRNA found in a wildtype animal. Thus, the deletion as defined herein may result in a large portion of the gene being absent e.g. at least 10, 20, 30, 40, 50, 60, 70, 80 or 90% of the gene being absent. The larger the deletion event which is carried out, the more likely the deletion will have a null effect on the gene i.e. will render the gene inactive with either no mRNA being transcribed or where the transcribed mRNA is not translated into a functional protein product. As discussed in detail below, a gene deletion may be made in the present invention using the LoxP/Cre or FRT/FLP systems.

As noted above, such insertions or deletions may be made anywhere in the targeted gene (e.g. the Erf gene). In particular they may be made in an intron or exon, or expression control region. In certain preferred embodiments insertions or deletions may be made in one or more introns, e.g. in the first intron of the Erf gene.

A point mutation as discussed above, is also considered to be a genetic modification of the present invention. A point mutation refers to the modification of a particular nucleotide base at a particular position to another nucleotide base e.g. modification of A to T, C or G, modification of T to A, C or G, modification of C to A, T or G or modification of G to C, A or T. Preferably, such a point mutation or substitution may effect transcription of the gene e.g. if the point mutation is made to the gene promoter or the point mutation may result in the substitution of an amino acid residue for a different amino acid residue in the translated protein which may affect its activity (this is referred to herein as a missense mutation). Further, the point mutation may code for a stop which can result in the production of a truncated protein. Such a point mutation is also referred to herein as a nonsense mutation.

Although the point mutation may result in a conservative amino acid change in the translated protein, preferably, the point mutation in the gene results in a non-conservative amino acid substitution in the final protein. Such non-conservative amino acid substitutions include substituting any of the amino acids in anyone of the following groups with an amino acid from another group:
1) glycine, alanine, valine, leucine, isoleucine
2) serine, cysteine, threonine, methionine
3) proline
4) phenylalanine, tyrosine, tryptophan
5) histidine, lysine or arginine
6) aspartic acid, glutamic acid, asparagine, glutamine.

Additionally, a silent point mutation which does not result in any amino acid change in the translated protein may be present in the gene, although at least one further mutation should be present in this case which is able to reduce Erf expression level and/or ERF activity.

As discussed above, several Erf mutations have been identified in human patients which are capable of causing ossification defects. In a preferred embodiment of the invention, the non-human transgenic animal comprises one or more of the mutations found in the human patient cohort which were associated with disease. The mutations in the non-human transgenic animal may occur at an equivalent position in the Erf non-human animal gene sequence to their position in the human Erf gene sequence. The human Erf mRNA transcribed from the human Erf gene has a nucleic acid sequence as set forth in SEQ ID NO. 4. The sequence of human ERF protein is as set forth in SEQ ID NO. 3. A genomic sequence showing the nucleotide sequence of the human Erf gene along with some flanking sequence is shown in SEQ ID NO. 8. The full length of the sequence shown in SEQ ID NO. 8 is 1-9593 nucleotides and the human Erf gene sequence lies at nucleotides 1001-8593 of SEQ ID. NO. 8 (mRNA is join (1001 . . . 1180, 5593 . . . 5827, 6216 . . . 6331, 6420 . . . 8593) and the CDS is join (1159 . . . 1180, 5593 . . . 5827, 6216 . . . 6331, 6420 . . . 7693)). The human Erf gene and ERF protein sequences are freely available and may be obtained from publically available databases (e.g. Genbank). Thus, particularly, the non-human transgenic animal may comprise a mutation at one or more positions equivalent to positions 547, 1512, 891, 892, 256, 194, 3, 1270, 21, 1201 and/or 1202 in the human cDNA sequence. Preferably, the non-human transgenic animal may comprise a mutation equivalent to 547° C.>T, 1512 deletion of T, 891-892 deletion of AG, 256° C.>T, 194 G>A, 3 G>A, 1270 OT, 21 A>T or 1201-1202 deletion of AA in the human Erf cDNA. Equivalent positions in the non-human animal Erf gene or cDNA sequences may be identified by optimally aligning the non-human animal Erf gene or cDNA sequences with the human sequence and identifying common or homologous regions.

As discussed previously, although a genetic modification as described herein may be made directly to Erf to effect its expression and/or ERF activity, it is also within the scope of the invention to make any other modifications which would have the desired effect. Thus, particularly, at least one modification (e.g. at least one insertion, deletion, inversion, substitution and/or point mutation as described above) may be made to any part of any other gene whose protein product would have an effect of the activity of ERF and/or on the expression of Erf. Modifications can therefore be made to genes whose protein products directly interact with ERF or which have a direct role in the transcription of Erf. Alternatively or additionally, modifications can be made to genes whose protein products indirectly have an effect on ERF activity and/or Erf transcription. Preferably, the modification(s) may cause an ossification defect. Preferably a modification is not made to FGFR2, FGFR3, TWIST1, EFNB1 and/or ERK.

Particularly, where a gene and/or its encoded product is known to have a positive effect on Erf expression and/or on ERF activity, the one or more modifications made to that gene in the scope of the present invention should result in a decrease in gene expression (and thus a decrease in the amount of protein present) and/or a decrease in protein activity. Such a decrease in the amount and/or activity of that protein would function to reduce Erf expression and/or ERF activity to the desired extent e.g. to less than 50% of the levels/activity seen in a wildtype animal and/or to produce an ossification defect. In this aspect, preferably, the one or more modifications would result in a decrease in gene expression and/or protein activity of at least 10, 20, 30, 40, 50, 60, 70, 80 or 90%. The modifications may result in a knockout of the gene if this does not result in death of the animal.

Particularly, where a gene and/or its encoded product is known to have a negative effect on Erf expression and/or on ERF activity, the one or more modifications made to that gene in the scope of the present invention should result in an increase in gene expression (and thus an increase in the amount of protein product present) and/or an increase in protein activity. Such an increase in the amount and/or activity of that protein would function to reduce Erf expression and/or ERF activity to the desired extent e.g. to less than 50% of the levels/activity seen in a wildtype animal and/or to produce an ossification defect. In this aspect, preferably the one or more modifications would result in an increase in gene expression and/or protein activity of at least 10, 20, 30, 40, 50, 60, 70, 80 or 90%.

As discussed above in relation to Erf gene modifications, the one or more modifications made to any other gene for the reduction of Erf expression and/or activity may be made to one or both alleles of the gene. Thus, any type of genotype modification may be made to either allele and to any part of that allele, providing that the modification results in the ultimate reduction of Erf expression and/or ERF activity. Thus the one or more modifications of the other gene(s) may result in an increase or decrease in expression levels and/or activity of the translated protein, depending on the effect that the wildtype protein product of that gene normally has with Erf/ERF.

The invention further encompasses reducing the Erf expression levels and/or ERF activity by increasing the expression or amount of other genes/proteins which have a negative effect on ERF or on Erf expression. In this respect, it is possible to achieve a reduction in ERF activity for example by increasing the number of copies of genes in an animal which encode for negative regulators of ERF. For example, a non-human transgenic animal comprising one or more additional copies of a gene encoding an Erk1/2 activator would be expected to have reduced ERF activity, in view of the increased phosphorylation of ERF and its removal from the nucleus. One or more additional transgenes can be included in a non-human animal where the gene (and more particularly its encoded protein) would have a negative effect on Erf expression and/or ERF activity. Further, Erf expression may be reduced in a transgenic non-human animal of the present invention by modifying the genome of the animal to express one or more inhibitory molecules, that is to express a nucleic acid molecule which may act to inhibit (or reduce or suppress etc.) expression of the ERf gene, for example an inhibitory RNA molecule. Accordingly, transgenesis of the animal with small nucleic acid molecules may be used to reduce Erf expression, e.g. with siRNA, siNA, dsRNA, miRNA or shRNA. siRNAs which could be used to reduce Erf expression levels and/or ERF activity are available from Santa Cruz Biotechnology, Inc (codes sc-43754 and sc-144923). shRNA plasmids are also available (code sc-43754-SH), as are shRNA lentiviral particles (code sc-43754-V).

Antisense may also be used as a means of reducing ERF expression. Thus, nucleic acids may be introduced into the animals which express, or allow to be expressed RNA molecules which act to inhibit, reduce or suppress Erf expression. Further, such RNA molecules or small nucleic acid molecules may be used which reduce the expression of any other gene whose protein product may activate, allow or upregulate Erf expression. The small nucleic acid molecules referred to above can be used to mediate RNA interference. siRNA are a class of double stranded RNA molecules that may be from 18 to 25, e.g. 19, 20, 21, 22, 23, 24 nucleotides in length.

A combination of any of the above described mutations or transgenesis approaches may be used in the non-human transgenic animal referred to herein. Thus, any one or more of the genes described above may be modified and/or the animal may comprise any one or more of the exogenous nucleic acids and/or genes discussed above. The only requirement is that the resulting non-human transgenic animal has a reduced level of Erf expression and/or ERF activity e.g. to less than 50% of that seen in a wildtype animal, and preferably that the transgenic animal has an ossification defect.

Thus, as discussed above, the non-human transgenic animal referred to herein may comprise many different types of modifications. However, a very similar method can be applied to produce a non-human animal comprising any one or more of the modifications and such methods of producing non-human transgenic animals are well known in the art.

Particularly, the non-human transgenic animals described herein may be produced by introducing an exogenous recombinant construct into an animal. Such a construct may comprise the nucleotide sequence of the gene into which it is desired to introduce a modification, or a portion of that gene (e.g. at least 5, 10, 15, 20, 25, 30, 35, 40, 45, 50, 55, 60, 65, 70, 75, 80, 85, 90, or 95% of the nucleotide sequence of the gene), where the gene or portion of the gene in the construct typically comprises the desired modification to be made. As discussed previously, such a modification can be an insertion, deletion, substitution, inversion, and/or point mutation. Thus, preferably, the gene or partial gene sequence in the construct comprises at least one modification.

Alternatively, where it is desired to increase the copy number of a particular gene in the transgenic animal, the exogenous recombinant construct may comprise one or more copies of the gene sequence without modification or with only modifications which preferably do not affect the function of the translated protein product e.g. silent point mutations or mutations which result in only conservative amino acid substitutions in the translated protein.

Where it is desired for the transgenic non-human animal to express a small nucleic acid molecule e.g. siRNA, then the exogenous recombinant construct may comprise a sequence which encodes such a nucleic acid molecule.

The exogenous recombinant construct may further comprise a promoter e.g. for the expression of the gene sequence or partial gene sequence where appropriate, for the expression of a small nucleic acid molecule or for the expression of another exogenous gene or nucleic acid sequence which may also be present in the exogenous recombinant construct and which may not have any effect on the expression level of Erf and/or activity level of ERF. In connection with this, the exogenous recombinant construct may comprise a marker gene e.g. an antibiotic resistance gene such as neomycin. Such a further gene may be found at any position in the construct e.g. adjacent to or within the nucleotide sequence of the gene into which it is desired to introduce a modification. If the nucleotide sequence of the further gene e.g. the marker gene occurs within the nucleotide sequence of the gene into which it is desired to introduce a modification, the presence of the further gene itself (and any promoter which is operably linked thereto) may constitute the modification i.e. the further gene and its promoter may be considered to be an insertion in the gene of interest.

In a preferred embodiment, the exogenous recombinant construct may comprise one or more recombinase sites or site-specific recombination sequences. These sites or sequences are recognised by a recombinase enzyme in facilitating a recombination event. Such sites or sequences may be wildtype or may comprise mutations, as long as functionality is preserved and the recombinase enzyme is able to recognise such sites to achieve recombination.

Preferably, the exogenous recombinant construct may comprise two site-specific recombination sequences, which in a particularly preferred embodiment may flank the modified gene/partial gene sequence, the unmodified gene sequence or the nucleotide sequence encoding a small nucleic acid sequence. Alternatively, the site-specific recombination sequences may flank any insertion modification introduced into the gene or partial gene sequence in the construct e.g. if it is desired at any point to remove such an insertion or the site-specific recombination sequences may flank any promoter or any other nucleotide sequence which it may be desired to excise, insert or recombine.

Examples of site-specific recombination sequences which may be present in the exogenous recombinant construct include LoxP and FRT sites or functional mutants of these sites e.g. those with at least 90 or 95% identity to SEQ ID NOs 5 or 6 as set out below.

The LoxP site generally comprises the 34 nucleotide base sequence of:

(SEQ ID NO. 5)
ATAACTTCGTATAGCATACATTATACGAAGTTAT.

The FRT site generally comprises the 34 nucleotide base sequence of:

(SEQ ID NO. 6)
GAAGTTCCTATTCTCTAGAAAGTATAGGAACTTC.

Recombinase or site-specific recombinase refers to an enzyme that carries out site specific recombination to change the structure of DNA. Particularly, a site specific recombinase may catalyse the recombination of DNA between the site-specific recombination sequences in a DNA molecule. Typically, these sequences contain a specific binding site for the recombinase that surround a directional core sequence where recombination can occur. Where two such sequences are present in a DNA molecule, the site-specific recombinase may cleave the DNA at both sequences and the DNA strands are then rejoined with DNA ligase. The result of the recombination generally depends on the orientation of the site-specific recombination sequences. For two sequences on the same chromosome arm, inverted sites may cause an inversion of the intervening DNA while a direct repeat of the sequences will cause a deletion of the intervening DNA. If site-specific recombination sequences are present on different chromosome arms, it is possible for translocation events to occur. Such enzymes particularly include site-specific recombinases and may further include transposases and lambda integration/excision systems. Systems which use well known recombinases include Cre-lox, FLP/FT, R/RS, Gin/gix, pSR1 system, cer system and fim system. Further systems have been identified in microorganisms such as phage, bacterium and yeast, including the E. coli lambda att P system for integration and excision and the Streptomyces phage C31 integrase.

The transgenic non-human animal discussed herein may therefore further comprise a nucleotide sequence which encodes a recombinase e.g. Cre or FLP. Such a nucleotide sequence may be under the control of a promoter which either allows ubiquitous or continuous expression of the recombinase, or alternatively, the nucleotide sequence encoding the recombinase may be under the control of an inducible promoter e.g. one which is activated by a particular stimulus. The use of an inducible promoter system may allow control of the expression of recombinase and thus control over any recombination event which the recombinase may perform e.g. any deletion event in the integrated exogenous recombinant construct.

Transgenic non-human animals as referred to herein may be produced by introducing an exogenous recombinant construct as discussed above into the germline of the non-human animal. Embryonal target cells at various developmental stages may be used to introduce the construct and different methods may be employed, depending on the developmental stage of the embryonic target cell. The methods include microinjection of zygotes, viral integration and transformation of embryonic stem cells. Procedures for producing transgenic animals are well known and widely described in the art and any convenient or desired procedure may be used.

The non-human transgenic animals described herein may be particularly produced by the transfomation of embryonic stem cells from the animal of interest with an exogenous recombinant construct described above. Marker-containing (e.g. drug resistant) embryonic stem cell clones obtained after transfection may be isolated and analysed for the present of the construct. A chimeric non-human animal may then be produced by the microinjection of a blastocyst of that particular animal species with an embryonic stem cell clone which has tested positive for the presence of the construct. Typically, the blastocyst may be 2, 3, 4 or 5 days post conception and preferably may be 3.5 days post conception. The injected blastocyst may then be implanted into a pseudopregnant non-human animal of the same animal species and the progeny are analysed to identify any animals comprising the integrated construct. Typically, as discussed above, any animals comprising the integrated construct at this stage may be chimeric i.e. some cell types of the animal may contain the construct and others will not. When producing transgenic mice, chimeric animals can be easily identified if the transfected embryonic cell clones used are from a mouse with a different coat colour to the mouse from which the blastocyst is taken. In this way, chimeric animals comprising the construct may be identified by the animal having fur patches of different colours. A chimeric animal which contains the integrated construct in its germline cells may then be selected for further breeding in order to produce a non-human transgenic animal heterozygous for the integrated construct. Typically, a chimeric animal is then mated with a wildtype animal and the resulting progeny are analysed to determine which comprises the introduced construct.

Further matings may be carried out to obtain non-human animals which are homozygous for the introduced (e.g. integrated) construct or to obtain non-human animals which have different introduced constructs. Additionally, further matings may be carried out to obtain non-human animals which further comprise a gene encoding for a recombinase. It will be appreciated to a skilled person, that the combination and number of matings which should be carried out will depend on the desired final genotype of the non-human transgenic animal.

As discussed previously, the non-human transgenic animal may in one embodiment comprise one or more modifications to either or both Erf alleles, wherein the animal has reduced Erf expression levels and/or reduced ERF activity. Particularly, the non-human transgenic animal may comprise an insertion in one or both Erf alleles which results in the reduction of Erf expression and/or ERF activity. More particularly, the insertion may comprise a promoter operably linked to a marker gene e.g. a PGK promoter linked to a neomycin resistance gene. Further, although the insertion may be made at any position in the Erf gene, preferably, the insertion occurs in an intron. Two site-specific recombinase sequences may further be inserted into the one or more alleles of Erf, where preferably one site specific recombinase sequence is situated at the 5′ end of the promoter (the first stie) and the other site specific recombinase sequence (the second site) is downstream of the 3′ end of the marker gene i.e. there is intervening Erf nucleotide sequence between the 3′ end of the marker gene and the second site specific recombinase sequence. As discussed above, the site specific recombinase sequence may be LoxP or FRT or a functional mutant thereof, but preferably is LoxP.

The non-human transgenic animal described above comprising the insertion in one Erf allele may be produced by transfecting embryonic stem cells with an exogenous recombinant construct comprising the Erf gene with the promoter-marker gene insertion. As discussed above, this construct may further comprise two site specific recombinase sequences, one which is 5′ to the promoter and one which is downstream of the 3′ end of the marker gene, allowing the presence of intervening Erf gene sequence between the end of the marker gene and the site specific recombinase sequence. In a preferred embodiment, the Erf gene sequence which lies between the 3′ end of the marker gene and the site specific recombinase sequence comprises at least some exonic sequence i.e. comprises at least some sequence which encodes the ERF protein. The Erf sequence which lies between the 3′ end of the marker gene and the site specific recombinase site may therefore comprise one or more exons of Erf of may comprise a portion of an exon e.g. 5, 10, 20, 30, 40, 50, 60, 70, 80 or 90% of an exon.

In a further preferred embodiment, the site specific recombinase sequence which lies downstream of the 3′ end of the marker gene, may be positioned after the stop codon of Erf. In this way, the site specific recombinase sequence may not interfere with transcription or with the final ERF protein whilst it is present in the gene sequence. The site specific recombinase sequence which is 5′ to the promoter of the marker gene, preferably is situated in an Erf intron e.g. the same intron as the promoter-marker gene and is preferably directly adjacent to the 5′ end of the promoter.

Thus, transfection of ES cells with a construct as described above, allows a recombination event in some ES cells between the naturally occurring Erf gene and the Erf gene of the transfected construct. In this respect as discussed previously, some ES cells may contain one Erf allele having the modified Erf sequence of the construct i.e. Erf with a promoter-marker gene insertion and with the two site specific recombinase sequences, one of which is 5′ to the promoter and the other of which is downstream of the 3′ end of the marker gene. As discussed above, preferably the promoter-marker gene—first site specific recombinase sequence occurs in an Erf intron and preferably the second site specific recombinase sequence (downstream of the 3′ end of the marker gene) is positioned after the stop codon of Erf. The chimeric non-human animal which is produced having such a modified Erf allele is then further mated to produce non-human animals with are heterozygous for the insertion. Further matings can then be carried out to obtain animals which are homozygous for the insertion or to obtain animals which also comprise a recombinase transgene.

Hence, in one particular embodiment, the invention provides a non-human transgenic animal comprising an insertion of a promoter-marker gene cassette in one or both Erf alleles wherein the cassette is inserted into an intron of Erf and wherein said animal exhibits reduced expression levels of Erf and/or reduced ERF activity.

Preferably, two site specific recombinase sequences are further inserted into the one or more Erf alleles, where the first site specific recombinase sequence is 5′ to the promoter of the inserted cassette and the second site specific recombinase sequence is downstream of the 3′ end of the marker gene. More preferably, the first recombinase sequence is directly adjacent to the 5′ end of the promoter linked to the marker gene and the second recombinase sequence is located 3′ to stop codon of the Erf gene e.g. 16 base pairs 3′ of the stop codon.

In a most preferred embodiment, the promoter may be a PGK promoter, the marker gene may be a neomycin resistance gene and/or the site specific recombinase sequences may be LoxP sites. Further, preferably, the promoter-maker gene cassette (and optionally the first site specific recombinase sequence) is inserted upstream of Erf exon 2 i.e. within Erf intron 1 e.g. 350 base pairs 5′ of Erf exon 2.

Further, in a preferred embodiment, both the first and second site specific recombinase sequences are inserted in the same orientation in the Erf gene.

Non-human transgenic animals which comprise the above described insertion in either allele where the insertion comprises the two site specific recombinase sequences in the same orientation may further be treated with recombinase (i.e. contacted with or exposed to) to delete the nucleotide sequence which lies between the two site specific recombinase sequences. Therefore, in an animal which comprises the insertion on only one Erf allele, the treatment with an appropriate recombinase may result in the production of an animal with a deletion in the Erf gene. The effect of the deletion on Erf expression and/or ERF activity will depend on the exact location of the inserted promoter-marker gene cassette and the location of the two site specific recombinase sequences. However, when the first site specific recombinase sequence occurs in an Erf intron and the second site specific recombinase lies downstream of the 3′ end of the marker gene with at least some intervening exonic Erf gene sequence, then treatment with an appropriate recombinase will allow the deletion of at least some Erf exonic sequence. When the second site specific recombinase sequence lies 3′ to the stop codon of the Erf gene, and the inserted cassette lies in one of the introns of Erf, then treatment with an appropriate recombinase will result in the deletion of at least one exon of Erf. Deletions of this size may result in a Erf null genotype from that allele i.e. no functional ERF protein may be produced from that allele. It is therefore possible to use the non-human transgenic animals with the insertions described above to produce animals which have an Erf^{+/-} genotype i.e. animals which have one functional allele of Erf and one non-functional allele of Erf.

The treatment of the animal with recombinase (for example and animal with the heterozygous insertion) may be carried out in practice by either mating the animal with another animal which carries a transgene coding for the recombinase and hence producing animal progeny with e.g. both the heterozygous Erf insertion and recombinase transgene, or by inducing expression of a recombinase transgene which is already present in that animal.

In a further preferred embodiment of the invention, the non-human transgenic animal comprises one null allele of Erf and one allele of Erf which has one or more modifications, resulting in the animal exhibiting a reduced level of Erf expression and/or ERF activity compared to a wildtype animal. Preferably, the animal has an ossification defect and/or has a level of expression of Erf and/or ERF activity which is between 5-50% of that of a wildtype animal, and/or exhibits a level of Erf expression and/or ERF activity which is less than 50% of the level of Erf expression and/or ERF activity of a wildtype animal (e.g. 5-49%, or any of ranges indicated above). In this embodiment, the null Erf allele does not produce any functional ERF protein and the Erf allele comprising one or more modifications preferably allows transcription/translation to produce a functional ERF protein, although at reduced levels and/or reduced activity compared to a wildtype allele, as discussed previously. Typically, such a transgenic non-human animal with one non-functional Erf allele and one modified allele will have Erf expression and/or activity levels which are less than 50% of the expression/activity levels of a wildtype animal.

More particularly, the non-human transgenic animal of the invention comprises an insertion of a promoter-marker gene cassette in one Erf allele wherein the cassette is inserted into an intron of Erf and a null mutation in the other Erf allele, wherein said animal exhibits reduced expression levels of Erf and/or reduced ERF activity.

Thus, in this embodiment one allele of the Erf comprises the insertion described above and the other allele has a null mutation. The allele comprising the null mutation may be the result of treating an allele comprising the insertion and the site specific recombinase sequences with recombinase or alternatively may be the result of making a different modification to the allele resulting in no ERF functional protein being obtained from that allele.

Such a transgenic animal may be produced by producing a transgenic animal as described above which comprises one wildtype Erf allele and one Erf allele comprising the promoter-marker gene cassette and two site specific recombinase sequences which may flank the cassette. Particularly, the first site specific recombinase site may be 5′ to the promoter sequence and is preferably directly adjacent thereto and the second site specific recombinase site is preferably downstream of the 3′ end of the marker gene, where the nucleotide sequence between the 3′ end of the marker gene and the second site specific recombinase sequence may be at least one or part of an exon of Erf. As discussed in detail previously, most preferably, the second site specific recombinase sequence may be placed 3′ to the stop codon of Erf and the promoter-marker gene and first site specific recombinase sequence may be placed upstream of the 5′ end of exon 2 of Erf (i.e. in Erf intron 1). In a particularly preferred embodiment an animal is produced which has a Erf^{+/LoxP} genotype, where “LoxP” in this context indicates the presence of an insertion of a LoxP-PGK-neomycin resistance gene cassette in Erf intron 1 (350 base pairs 5′ of Erf exon 2) and a LoxP site 16 base pairs 3′ of the Erf stop codon. Once this heterozygous animal has been produced, it may be treated with recombinase as discussed previously to produce an animal with the Erf^{+/-} genotype where the nucleotide sequence between the two site specific recombinase sequences has been deleted. The animal with the Erf^{+/-} genotype may then be mated with an animal which is heterozygous for the insertion i.e. which has the Erf^{+/insertion} genotype (one Erf allele is wildtype (+) and one Erf allele has the insertion). As discussed above, preferably this animal has the Erf^{+/LoxP} genotype. This mating will result in some animals being produced which have the genotype of Erf^{insertion/-} (i.e. one allele of Erf has the insertion and one allele of Erf is null). Preferably, animals with the Erf^{LoxP/-} genotype will be produced. Similar matings may be carried out to produce animals which have any other null mutations made to one Erf allele and which have the insertion (or another modification which results in a reduced level of Erf and/or activity of ERF) in the other Erf allele.

Particularly, the Erf^{insertion/-} genotype animals of the invention e.g. Erf^{LoxP/-} have ossification defects and more particularly have craniosynostosis.

As indicated previously, the present inventors have found that modifications made to the Erf gene in transgenic mice which result in Erf expression levels being reduced to less than 50% of the expression levels seen in a wildtype animal can result in the animal showing ossification defects and particularly a domed head phenotype which is associated with craniosynostosis.

In the context of this work, the present inventors have produced transgenic animals with lower levels of Erf gene or ERF protein expression than previously reported. In particular it is believed that we have produced for the first time, transgenic animals which exhibit less than 50% Erf gene or ERF protein expression, compared to wild-type.

Accordingly, in another aspect, the present invention also extends to a non-human transgenic animal comprising a modification in its genome which results in the animal exhibiting a level of Erf expression and/or ERF activity which is less than 50% compared to a wildtype animal.

As noted above, in a preferred embodiment such a transgenic animal may show an ossification defect e.g. craniosynostosis.

As discussed herein, the non-human transgenic animal of the invention may have an ossification defect. The term “ossification defect” refers to any defect which occurs in the formation/growth pattern of the bones of an animal or in the timing of the formation of bones in an animal. Thus, any defect which results in any bones not having the correct positioning, size, structure, orientation or attachment to other components at a particular developmental stage may be classed as an ossification defect. Particular ossification patterns typically occur in animals at particular developmental stages and thus any differentiation from the normally observed ossification at a particular developmental stage is considered to be an ossification defect. An ossification defect may therefore be an increase (e.g. of at least 5, 10, 15, 20, 30, 40, or 50%) in ossification at a particular developmental stage compared to a normal animal at the same developmental stage.

Preferably, ossification defects described herein refer to defects of the cranium or of the mandible i.e. defects of the skull. However, other defects, such as brachydactyly (short fingers and/or toes) or broad fingers and/or toes are encompassed. Particularly, an ossification defect of the present invention may be craniosynostosis which is characterised by the premature fusion of one or more cranial sutures and/or may be Crouzon syndrome which affects the first branchial (or pharyngeal) arch of the maxilla and mandible. Craniosynostosis includes the premature fusing of a single suture in the cranium e.g. of the sagittal, coronal, metopic or lambdoid sutures, or the premature fusing of multiple sutures in the cranium i.e. any combination of more than one of the sagittal, coronal, metopic or lambdoid sutures. The premature fusing of one or more cranial sutures may have an effect on the shape of the head and/or face of animal and such animals may present for example with dome shaped heads. Hence, as the skull cannot expand perpendicular to any fused suture, it may compensate by increasing its growth in the direction parallel to the closed suture to provide space for the growing brain. Such a different ossification pattern can cause an abnormal head shape and facial features, as discussed above. Thus, an animal with craniosynostosis may have further phenotypic characteristics, e.g. may have increased intercranial pressure, a Chiari Type I (or Type II, III or IV) malformation where the cerebellar tonsils are displaced downward through the foramen magnum at the base of the skull, hyprtelorism, midface hypoplasia, exorbitism, macrocephaly and/or neurodevelomental delay e.g. delayed or reduced verbal ability, processing speed, intellectual ability, behavioural difficulties.

The non-human animal which is referred to herein may be any animal, although is preferably it is a vertebrate, and more particularly a non-human mammal, including in particular a rodent e.g. a mouse, or rat, a guinea gig, a cat, a dog, goat, sheep, pig, cow, a primate or a rabbit. In a particular embodiment, the non-human animal is a transgenic mouse.

As noted above, the term “transgenic” as used herein refers particularly to a non-human animal which comprises an exogenous nucleotide sequence i.e. a nucleotide sequence which has been artificially introduced, e.g. which is foreign to the animal or is non-native. In the case where modifications are introduced into one or both alleles of a gene of the animal, the exogenous nucleotide sequence may include the modified portion of the gene. Further, particularly in the instance where additional copies of a gene are introduced into the animal or where a nucleotide sequence encoding a small nucleic acid is introduced, the exogenous nucleotide sequence may include a promoter and/or a marker gene which are not naturally found in the native animal. However, an animal is considered to be transgenic if it comprises any nucleotide sequence which is non-native to the wildtype animal, or which is present in a non-native location. As also noted above, transgenic animals include those animals into which the exogenous nucleotide sequence is directly introduced or which were formed by any particular matings and further includes any animals derived (either directly or indirectly) from those animals. Thus progeny animals are included.

Whilst the primary use of the animals of the present invention which exhibit an ossification defect is of course as animal models for the disease, animals which have a genetic modification and which exhibit a reduced level of Erf expression and/or ERF activity but which may not have an ossification defect also have several important uses. Particularly, such animals may be used to produce transgenic animals which have an ossification defect. Further, such animals may be used to screen for drugs, compounds or treatments which may result in an increased level of Erf expression and/or ERF activity and which may be effective in treating an ossification defect associated with a reduced Erf expression level and/or a reduced ERF activity.

Thus, the invention extends to the use of a non-human transgenic animal comprising a genetic modification which results in the animal exhibiting a level of expression of Erf and/or ERF activity which is reduced compared to a wildtype animal in the production of a non-human transgenic animal which exhibits a reduced level of expression of Erf and/or ERF activity compared to a wildtype animal and which has an ossification defect. Hence, as indicated above, transgenic animals which have a genetic modification which although results in a reduced expression level of Erf and/or reduced ERF activity, may not always have an ossification defect. The level of Erf expression and/or ERF activity needs to be reduced to below a particular level in order for ossification defects to be detected. Thus, some modifications which may be made may not reduce Erf expression levels and/or ERF activity to below this threshold and such animals may not have an ossification defect. However, these animals may be valuable tools in producing animals which do have a ossification defect and matings of these animals may allow the production of such an animal having an ossification defect. For example, as discussed above, a transgenic non-human animal with a heterozygous modification (i.e. with one modified allele and one wildtype allele) but with no ossification defect phenotype may be mated with another animal e.g. with an animal having the same or a different heterozygous modification to produce animals which are homozygous for the mutation or which have compound modifications (i.e. different modifications to each allele). Such homozygous animals may have an ossification defect phenotype. Particularly, non-human transgenic animals having modifications to Erf e.g. the insertion discussed above, may be mated in this way and such animals having a normal ossification defect phenotype may be used to produce animals with ossification defects.

Accordingly it is preferred that the transgenic animal of the invention is fertile. However, in certain embodiments, it is not precluded that it is sterile.

Alternatively viewed, the invention provides a method of producing a non-human transgenic animal which exhibits a level of expression of Erf and/or ERF activity which is reduced compared to a wildtype animal and which has an ossification defect comprising mating a non-human transgenic animal comprising a modification in its genome which results in the animal exhibiting a level of expression of Erf and/or ERF activity which is reduced compared to a wildtype animal with a further non-human transgenic animal comprising a modification in its genome which results in the animal exhibiting a level of expression of Erf and/or ER activity which is reduced compared to a wildtype animal.

Additionally, as indicated above, non-human transgenic animals which have a genetic modification which results in reduced Erf expression level and/or in reduced ERF activity may be used to investigate treatments e.g. drugs or compounds which may increase Erf expression levels and/or ERF activity and which therefore may be of use in treating ossification defects associated with a reduced Erf expression level/ERF activity. Thus, the invention provides in this aspect, the use of a non-human transgenic animal with a genetic modification which results in a level of expression of Erf and/or ERF activity which is reduced compared to a wildtype animal to identify a test agent or treatment which increases Erf expression and/or activity and which may be capable of treating an ossification defect associated with a reduced level of Erf expression and/or ERF activity.

Alternatively viewed, the invention provides a method of identifying a test agent or treatment for increasing Erf expression levels and/or ERF activity which may be capable of treating an ossification defect associated with a reduced Erf expression level and/or ERF activity comprising administering said agent or treatment to a non-human transgenic animal as defined herein comprising a modification in its genome which results in the animal exhibiting a level of expression of Erf and/or ERF activity which is reduced compared to a wildtype animal and determining whether said non-human transgenic animal has an increased expression level of Erf and/or ERF activity after said treatment. Erf expression levels and ERF activity can be measured as discussed previously above. An increased level of Erf expression and/or activity of at least 10, 20, 30, 40 or 50% in said treated non-human transgenic animal may be indicative of a treatment or agent which can increase the level of expression of Erf and/or ERF activity and which may therefore be useful for treating an ossification defect.

Further, transgenic animals which have a genetic modification which results in the reduction of Erf expression and/or ERF activity but which do not have an ossification defect, may be of use as tools to determine whether any other phenotypic changes are associated with a reduced level of expression of Erf and/or reduced ERF activity.

The non-human transgenic animals of the invention which have an ossification defect have several important uses in the study of ossification defects and in the identification of a possible treatment for such ossification defects.

Thus, in one aspect the invention provides the use of a non-human transgenic animal comprising a genetic modification which results in said animal exhibiting a level of Erf expression and/or ERF activity which is reduced compared to a wildtype animal and which has an ossification defect, as a model to study ossification defects. The study of ossification defects in the animal models of the invention can enable important information to be gathered concerning the progression, prognosis, diagnosis and treatment of the disease condition. Thus, studying animal models which have a disease phenotype e.g. which have an ossification defect, can allow the determination of how certain disease phenotypes will progress and whether any treatment is required for particular disease phenotypes. Further different and appropriate treatments can be suggested for particular disease phenotypes by studying progression and prognosis of particular conditions.

Alternatively viewed therefore, the present invention provides the use of a non-human transgenic animal comprising a genetic modification which results in said animal exhibiting a level of Erf expression and/or ERF activity which is reduced compared to a wildtype animal and which has an ossification defect, as a disease model for ossification defects. Particularly, the invention provides the use of a non-human transgenic animal comprising a genetic modification which results in said animal exhibiting a level of Erf expression and/or ERF activity which is reduced compared to a wildtype animal and which has an ossification defect as a disease model for craniosynostosis.

As discussed previously, one particular use of the non-human animals of the invention is to screen for treatments e.g. drugs/compounds which may improve or lessen the ossification disease phenotype which occurs in the animals. Such identified treatments may then be investigated for their activity and ability to treat disease in other animals e.g. in human patients. In this aspect, the present invention provides the use of a non-human transgenic animal comprising a genotype modification which results in said animal exhibiting a reduced level of Erf expression and/or ERF activity compared to a wildtype animal and which has an ossification defect, to identify a treatment for the ossification defect. Particularly, the treatment may be a drug or a compound which improves the phenotypic presentation of the disease. The treatment e.g. drug or compound may be one which functions by increasing the level of expression of Erf and/or ERF activity.

Alternatively viewed, the invention provides a method of identifying a test agent or treatment for an ossification defect comprising administering said agent or treatment to a non-human transgenic animal comprising a modification in its genome which results in said animal exhibiting a level of Erf expression and/or ERF activity which is reduced compared to a wildtype animal and which has an ossification defect, and determining whether said treatment improves the ossification defect. An improvement of an ossification defect includes any improvement in the phenotypic presentation of disease e.g. where the defect is a result of an increase in ossification at a particular developmental stage, an improvement may consist of a reduction in ossification e.g. at the same developmental stage.

The invention further provides a method for producing a non-human transgenic animal as defined herein said method comprising introducing into the genome of said animal a genetic modification which results in said animal exhibiting a level of Erf expression and/or ERF activity which is reduced compared to a wildtype animal and which preferably causes said animal to display a defect of ossification.

Further included in the present invention is an additional step of generating progeny from said animal e.g. by breeding or mating said animal.

Finally, the present invention also extends to any cells or cell lines developed or isolated from a non-human transgenic animal of the invention.

Also particularly provided are transgenic gametes, including a transgenic ovum or sperm cell, a transgenic embryo, and any other type of transgenic cell or cluster of cells, whether haploid, diploid of higher zygosity, which comprise the genetic modification of the invention to reduce Erf expression or ERF activity, according to any aspect of the invention as described herein.

The invention will now be described in more detail in the following non-limiting Examples with reference to the drawings in which:

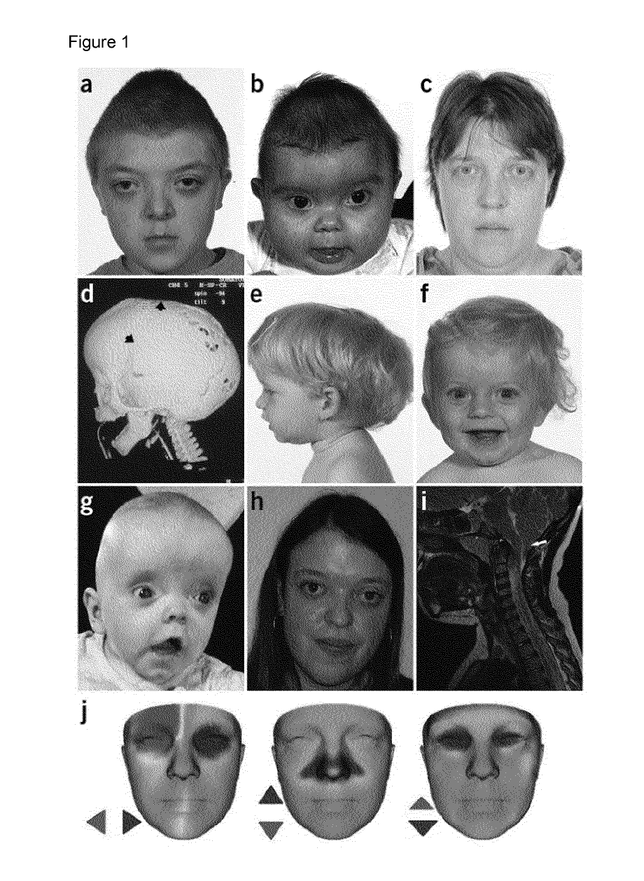

FIG. 1 shows Clinical features of subjects heterozygous for Erf mutations, (a-c) Family 1 showing proband IV-2 aged 4 months, in whom exome sequencing was performed (a), his brother IV-1 aged 10 yr (b) and mother III-3 aged 37 yr (c). (d-g) Subjects identified in follow-up sequencing had clinical diagnoses ranging from FGFR2 mutation-negative Crouzon syndrome (d, II-1 in Family 10 aged 4 months; e, III-I in Family 7 aged 18 yr) to non-syndromic sagittal (f, III-I in Family 5 aged 1.6 yr) or lambdoid synostosis (g, III-I in Family 4 aged 1.2 yr). (h) Computed tomographjc head scanning (IV-2 in Family 1 aged 5 months) showing synostosis of the left coronal and sagittal sutures (arrowheads) associated with multiple craniolacunae. The lambdoid and squamosal sutures remain patent, (i) Magnetic resonance brain imaging (sagittal, TI view) of III-I in Family 4 aged 7.1 yr, showing Chiari malformation with 12 mm herniation of cerebellar tonsils (arrowhead), (j) Comparison of average faces between EKF-mutant (n=14) and control (n=381) subjects. Red/blue denotes normalized displacement at over 1.5 SD, highlighting shared features of hypertelorism (left), vertical nasal displacement (centre) and prominent forehead with exorbitism (right). Written consent was provided for the publication of all photographs.

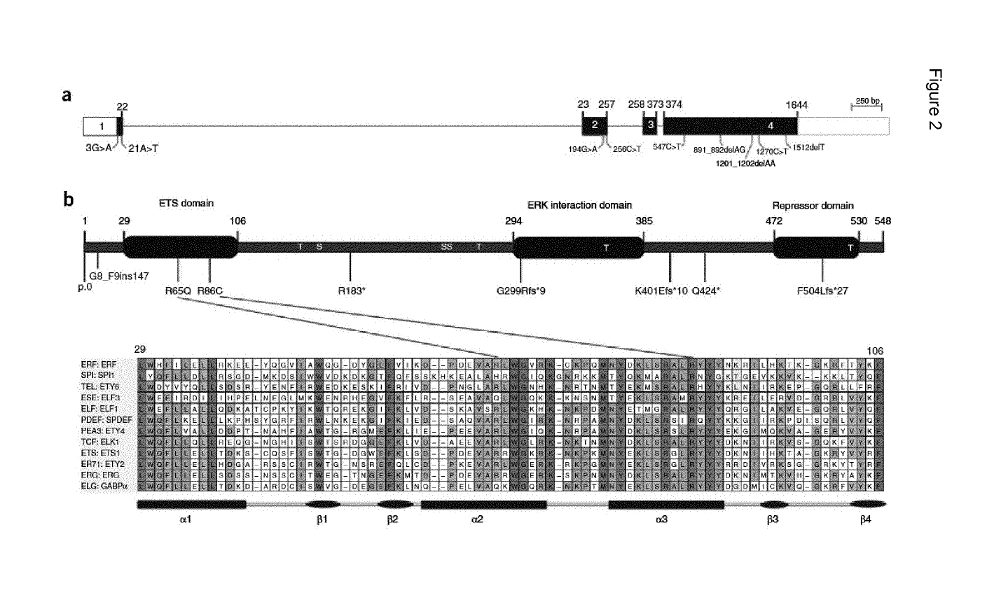

FIG. 2 shows Exon and domain structure of Erf and mutations identified in craniosynostosis. Erf comprises 4 exons (a) extending over 7.6 kb and encodes a 548 amino acid protein (b). Two missense mutations p. Arg65Gln and p.Arg86Cys localize to the ETS DNA-binding domain. The lineup in the bottom panel shows the ETS domain sequence in a representative member of each ETS subfamily from humans.

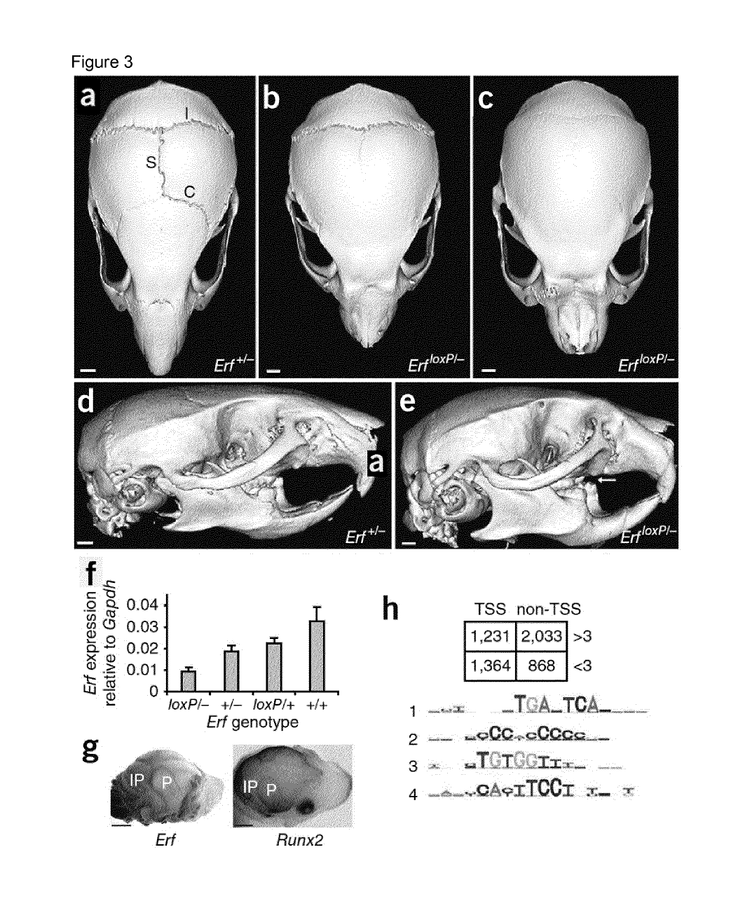

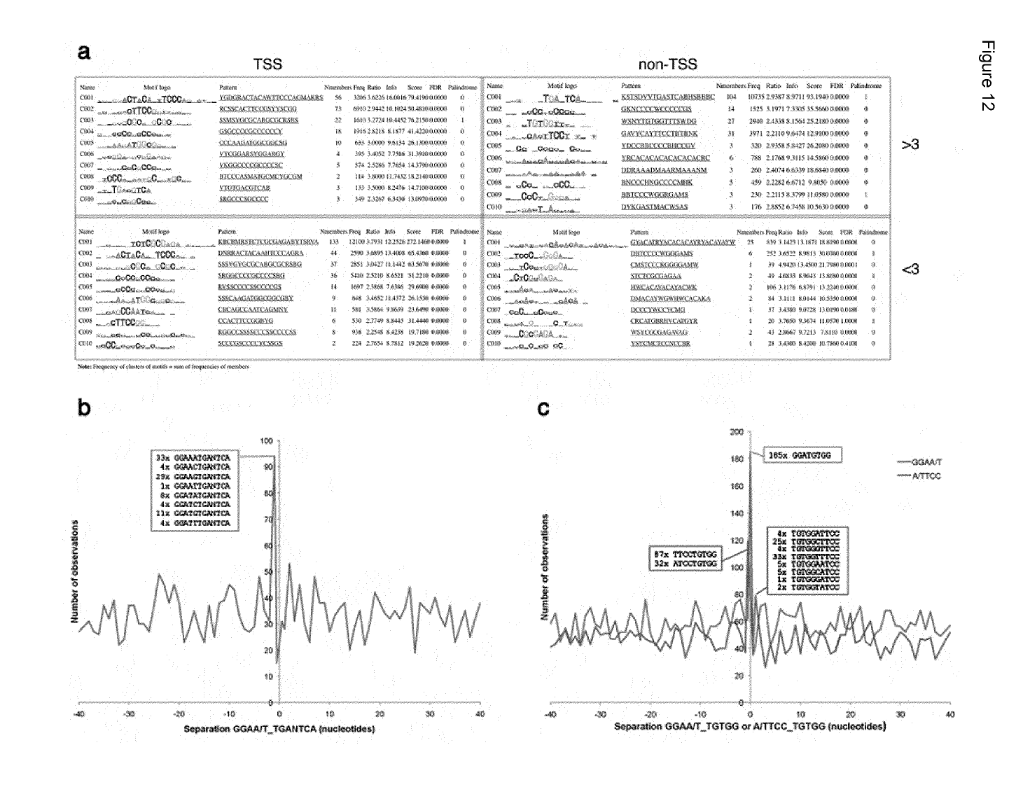

FIG. 3 shows analysis of Erf in mouse mutants and embryonic fibroblasts, (a-e), Micro-CT scanning of heads of mice aged 9 weeks. Note normal morphology and patent sagittal (s), coronal (c) and lambdoid (1) sutures in the Erf′ mutant (a,d), whereas the Erf^{loxP/-} littermates have craniosynostosis of the sagittal and coronal sutures (b) or sagittal, coronal and lambdoid sutures (c). Note dental malocclusion (arrow) on the side view (e) of skull shown in c. (f) Quantitative RT-PCR of Erf in EI 6.5 calvariae of different genotypes, showing reduced expression of Erf^{loxP} relative to wild type Erf allele. Error bars indicate standard error of mean, (g) RNA in situ hybridization of Erf (left) and Runx2 (right) in wild type EI 6.5 mouse calvaria. Note similar expression patterns coinciding with osteogenic fronts of calvarial bones, (h) Summary of ChIP-Seq analysis using antibody to ERF in mouse embryonic fibroblasts. In the upper panel, box shows number of peaks identified according to whether they located within 1 kb of a transcription start site (TSS) and whether they showed loss of binding in the presence of FCS (FCS−/FCS+>3). In the lower panel, MEME analysis of the 2033 non-TSS dynamically bound peaks (>3) identifies enrichment for motifs corresponding to binding sites for AP-1 (#1: 5′-TGANTCA), RUNX (#3: 5′-TGTGG) and ETS (#4: 5′-TTCCT). Motif #2 was also observed in TSS peaks (FIG. 12a).

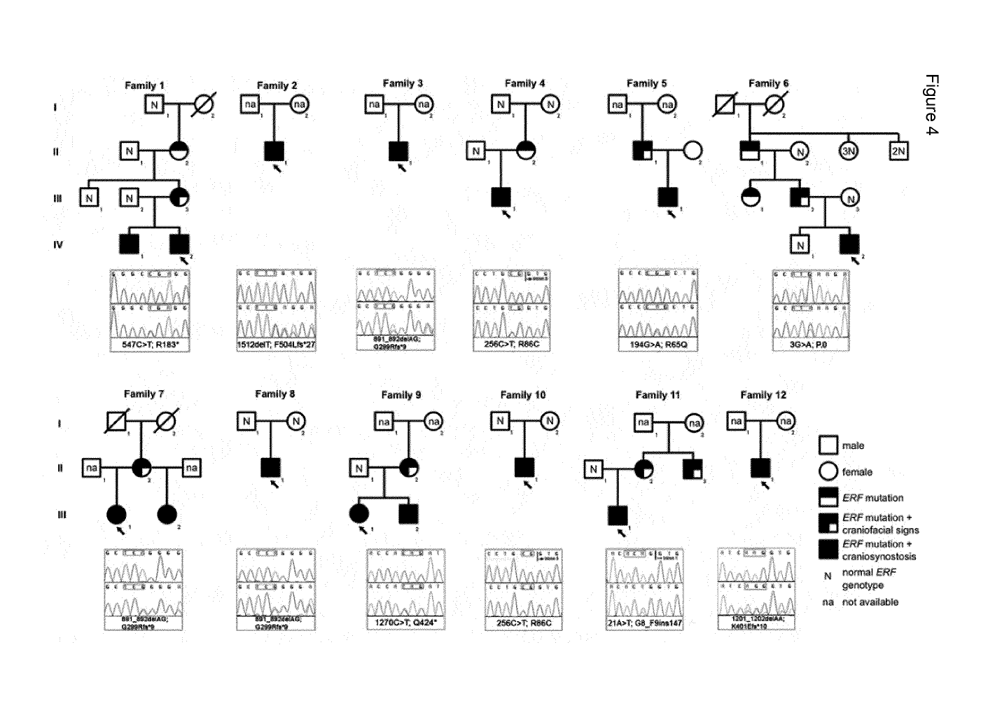

FIG. 4 shows pedigrees of 12 families with mutations in Erf and results of dideoxy sequencing.

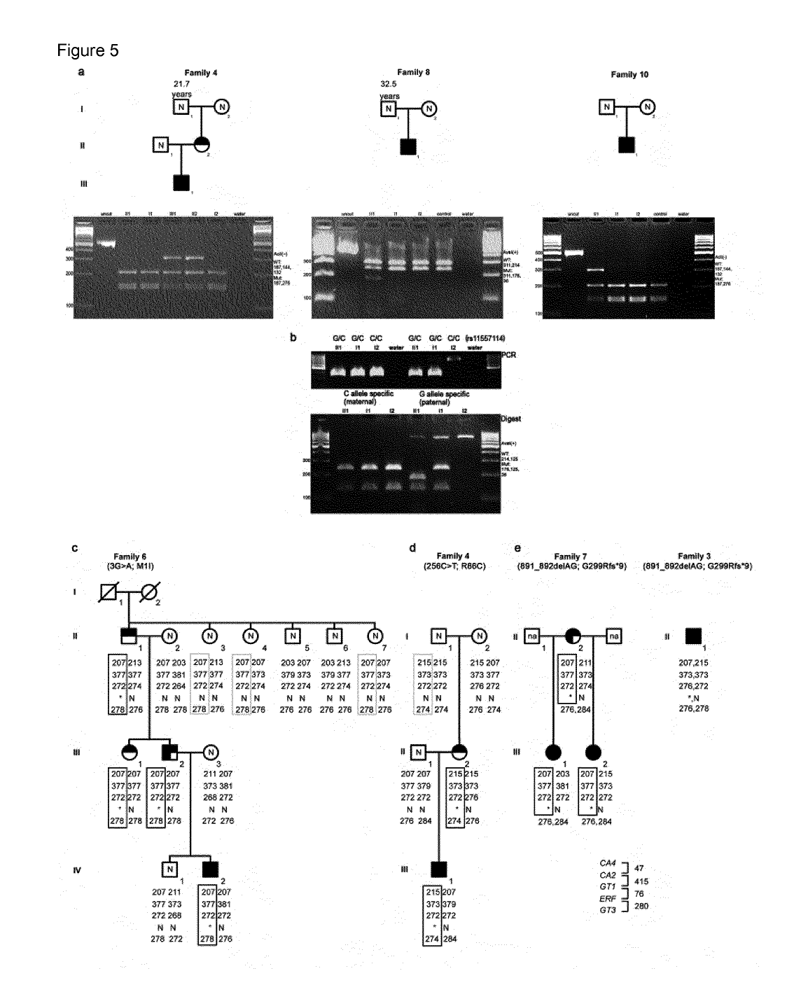

FIG. 5 shows analysis of de novo Erf mutations and independent origin of identical mutations. FIG. 5a-b show restriction digest analysis to demonstrate de novo mutations. For families 4, 8 and 10, diagnostic digests demonstrate absence of the mutation in either the parental (Families 8 and 10) or grandparental (Family 4) generation (upper panel). The lower panel (FIG. 5b) illustrates allele-specific PCR analysis of rs11557114° C./G SNP, demonstrating the paternal origin of mutation in Family 8 (in Family 4, microsatellite analysis was employed, see section d). Ages of unaffected fathers at birth of their affected child are indicated. FIG. 5c-e shows the analysis of chromosome segregation around Erf using 4 microsatellite markers. FIG. 5c shows that in Family 6, the mutation has arisen de novo in individual II-1 because 3 of his mutation-negative siblings (II-3, I-4, I-7) inherited the same chromosome that bears his Erf mutation. FIG. 5d shows that in Family 4, the mutation has arisen from the unaffected grandfather. FIG. 5e shows Families 7 and 3 with the same mutation have differing microsatellite genotypes at the CA2 locus. Disease haplotypes are boxed with black surrounding lines in individuals bearing the Erf mutation an gray surrounding lines in Erf mutation-negative subjects. The position of Erf in relation to the microsatellites (separation in kb) is shown in the key.

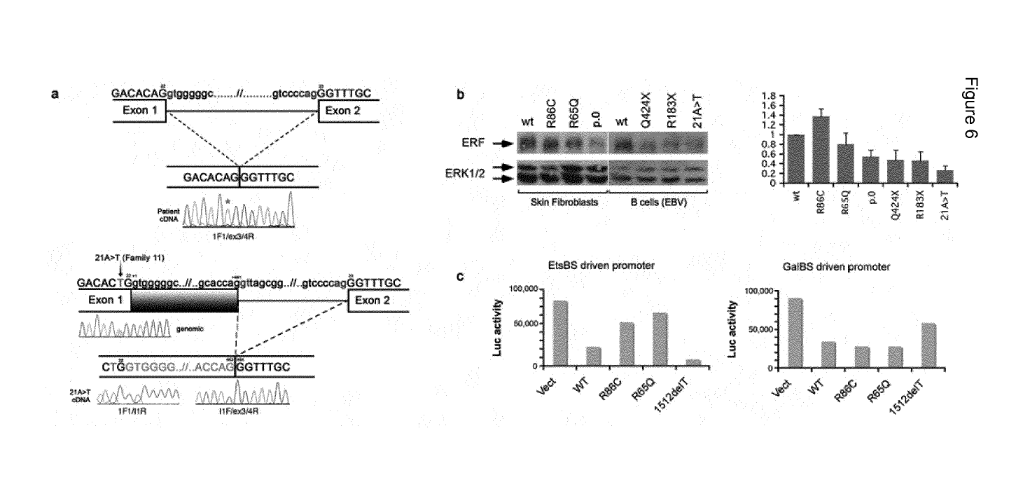

FIG. 6 shows RNA and protein studies of selected Erf mutations. FIG. 6a shows sequence analysis of lymphoblastoid cell line cDNA from individual III-1 (Family 11, FIG. 4, heterozygous for 21A>T mutation). The upper panel shows the result of RT-PCR using pimers located in exon 1 and exon 3/ (1° F.1 and ex3/4R; see Table 6): only the normal A allele (red asterisk) is visualised, indicating complete loss of the mutant T allele in the normal mRNA. In the lower panel, the intron 1 sequence contains a potential cryptic donor splice site starting at nucleotide 442. RT-PCR using primers in intron1 (reverse primer I1R combined with 1F1, and forward primer I1F combined with ex3/4R), demonstrate that the use of this cryptic splice site occurs exclusively on the allele harbouring the 21A>T mutation. FIG. 6b shows Western blot analysis of selected Erf mutations in fibroblasts or lymphoblastoid cell lines. Reduced amounts of ERF are produced in cells expressing disruptive mutations (M1 L, Q242X, R183X, 21A>T). The quantification in the right hand panel shows the mean+/−SD compared to wildtype. FIG. 6c shows DNA-binding domain mutants fail to repress Ets-binding site (EtsBS)-containing promoters (left) but function when tethered to the gal4-DNA binding domain on gal4 binding site (GalBS)-containing reporters (right). Conversely, the deletion/frame-shift mutation failed on gal4 but worked on the EtsBS promoter; this is expected as the ets-DNA binding domain, when overexpressed, is sufficient to out compete endogenous factors.

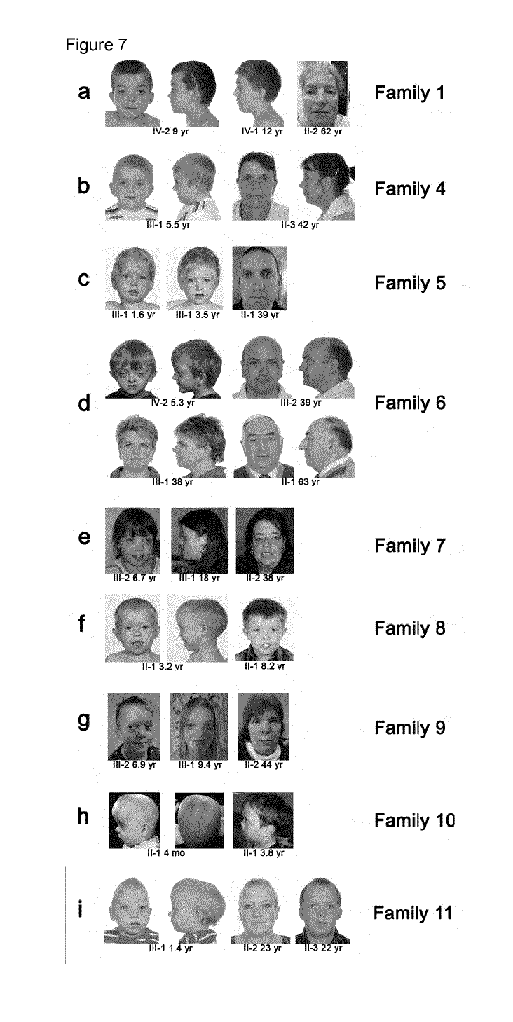

FIG. 7 shows a survey of facial features in subjects with Erf mutations. Each row (a-i) shows individuals from a different family. Written consent was provided for the publication of all photographs.

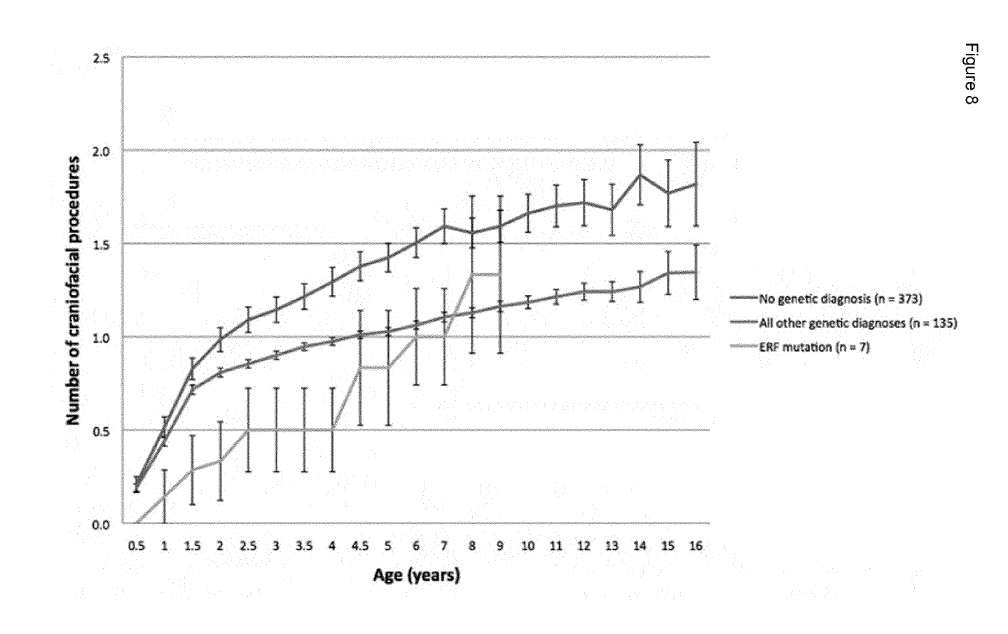

FIG. 8 shows the timing of major craniofacial surgery in Erf mutation-positive individuals with craniosynostosis. Points are plotted as +/− standard error of mean, of number of major craniofacial procedures undertaken at a given age. Comparative data with cases either having a different genetic diagnosis, or no genetic diagnosis made, are for Oxford patients born between 1993 and 2006. Data for Erf are not plotted beyond age 9 years because insufficient subjects were available (n<6).

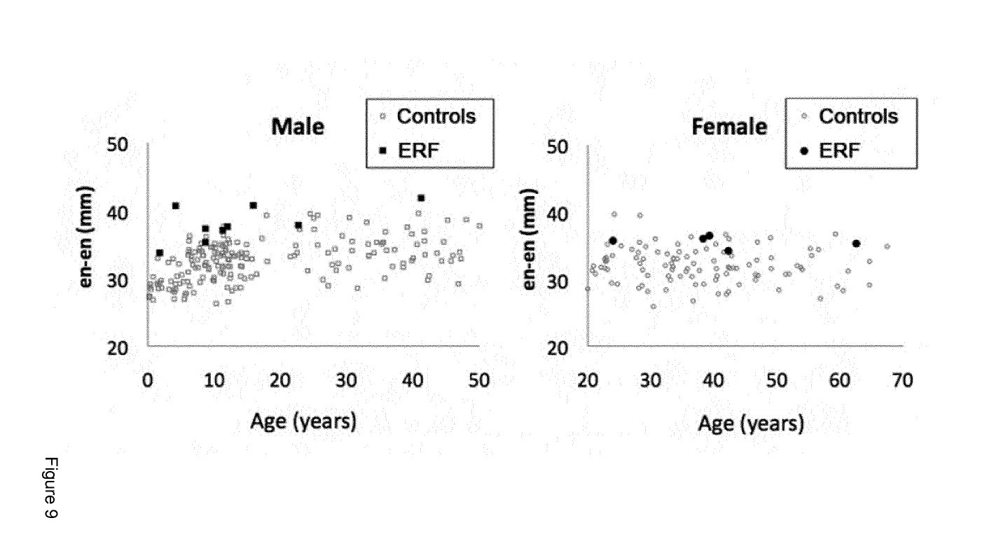

FIG. 9 shows inner canthal separation (en-en) determined from facial imaging (of 14 Erf mutation-positive individuals). Hypertelorism is highly significant in both affected females (p<0.00005) and affected males (p<0.0005).

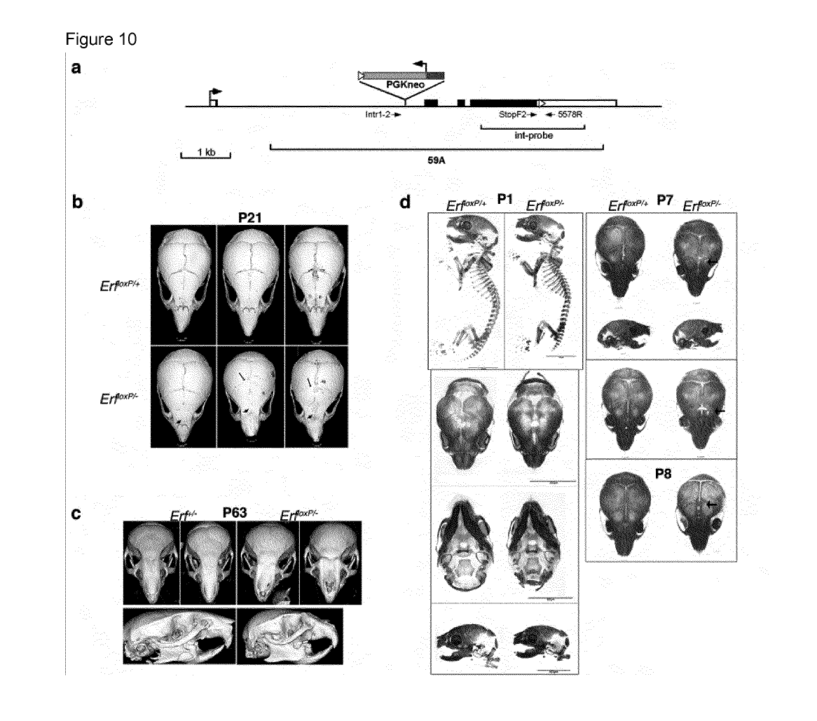

FIG. 10 shows construction and characterization of the murine Erf^{loxP} allele, (FIG. 10a), Schematic representation of murine Erf gene and genomic region used for targeting (59A). The black boxes indicate the Encoding regions, white boxes the 5′ and 3′-untranslated regions, unfilled triangles the loxP sites, the dark and light gray boxes the PGK promoter, and neomycin-resistance gene, respectively. Positions of the 2.2 kb BamHI-XbaI fragment (int-probe) and the PCR primers used are indicated. FIG. 10 b, shows micro-CT images of the skull at various ages. Representative Alizarin red/Alcian blue-stained preparations at P1 (left) and P7/8 (right). At P1 note smaller but proportionate skeleton of Erf^{LoxP/-} animal compared with Erf^{LoxP/+} littermate (top; whole skeletons are composite images). No craniosynostosis is evident at this stage; 5 Erf^{LoxP/-} and 3 Erf^{LoxP/+} were examined. By P7, skulls of Erf^{LoxP/-} animals are noticeably shorter and domed but craniosynostosis is not evident; in addition, most Erf^{LoxP/-} mutants showed an irregular margin of the medial part of frontal bone with reduced mineralisation (arrows). Eight Erf^{LoxP/-} and 11 Erf^{LoxP/+} were examined. FIG. 10c shows skeletal preparations (alizarin red/alcian blue staining) at various ages. These are micro-CT scans of P21 skulls from Erf^{LoxP/+} (top) and Erf^{LoxP/-} (bottom) littermates. Note that coronal craniosynostosis is beginning to be apparent in 2 of 3 Erf^{LoxP/-} animals (arrows). The nasal bones (arrowheads) are already abnormal in all three Erf^{LoxP/-} animals, associated with simplified morphology of the frontonasal suture. FIG. 10d shows micro-CT scans of P63 skulls from 2 Erf^{+/-} and 2 Erf^{LoxP/-} littermates (3 of these mice are also illustrated with different views in FIG. 3a-e). Top, frontal views of all four mice (Erf^{+/-} on left is not shown in FIG. 3). Note twisted broad snouts and telorbitism of Erf^{LoxP/-} mutants. Bottom, side views of 2 mice not shown in FIG. 3d-e.

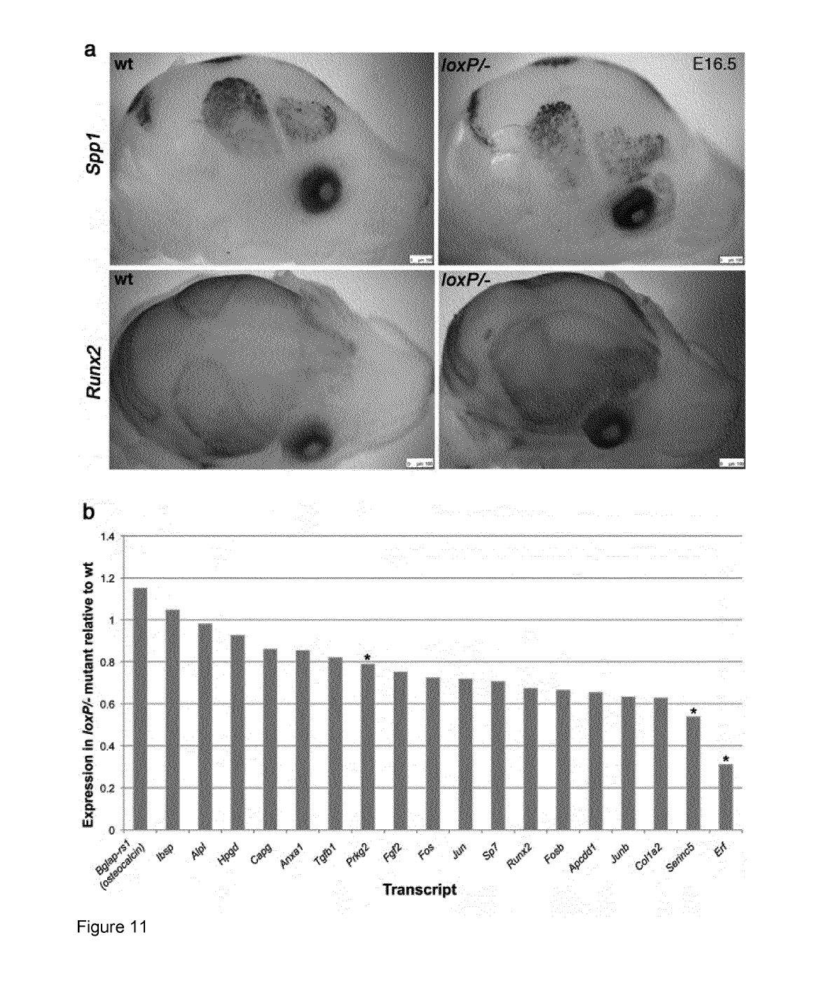

FIG. 11 shows additional in situ images and real time RT-PCR expression data. FIG. 11a shows expression of osteopontin (Spp1) and Runx2 in calvaria of representative E16.5 wildtype (WT) and Erf^{LoxP/-} embryos. No consistent differences in expression for a given gene were detected between WT and mutant embryos. FIG. 11b shows real time PCR analysis of Erf and 18 osteogenic marker transcripts in dissected EI 6.5 calvaria. Data are based on comparison of wild type (n=5) and Erf^{LoxP/-} (n=4) mice from two litters. The average ratio of expression in the mutants to the wild types is shown, asterisks indicate differences significant at P=0.05 level (t-test).

FIG. 12 shows analysis of ChIP-seq data (a) Summary of MEME patterns. (b,c) Spatial relationship of consensus transcription factor binding sites between ETS and RUNX (b) and ETS and API (c). FIG. 12a shows MEME analysis of sequence under CHIP-seq peaks within 1 kb of transcription start sites (TSS) or not (Non-TSS) and with a −FCS/+FCS ratio of greater or less than 3. FIG. 11b-c show plots showing enrichment, at particular separations, of adjoining ETS and AP1 binding sites b-c, plots showing number of sequences observed, at particular separations of closely spaced ETS and AP1 binding sites (b) and ETS and RUNX sites (c), in peaks defined as non-TSS and FCS−/FCS+>3. The x-axis shows the number of nucleotides separating the two motifs, except that in both plots, abutting motifs are plotted at −0.5 and +0.5, and in plot c, the specific overlapping motif GGATGTGG is plotted at 0. Sequence compositions of peaks representing closely adjacent motifs are shown individually. Owing to palindromic nature of AP1 site only a single plot is shown, whereas for the ETS-RUNX pairing the two possible orientations are shown separately.

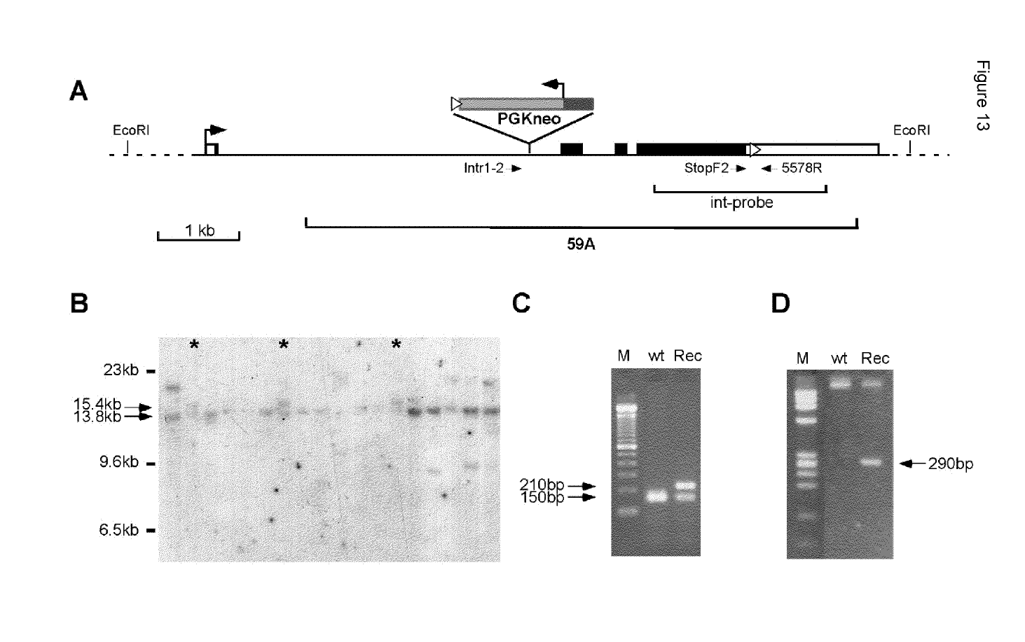

FIG. 13 shows in A, a schematic representation of the targeting allele. The black boxes indicate the Erf coding regions; white boxes the Erf 5′ and 3′-untranslated regions; triangles indicate the loxP sites; dark grey boxes the PGK promoter region; light grey boxes the neomycin resistance gene coding sequence. The position of the probe and PCR primers used in (B), (C) and (D) are also indicated. 59A indicates the genomic region used for targeting. Dashed lines are not in scale. FIG. 13B shows a Southern blot of genomic DNA from individual clones digested with EcoRI and hybridised with the 32P-labelled 2.2 kb BamHI-XbaI fragment (int-probe). Asterisks indicate clones with homologous recombination and absence of additional insertions. Arrows indicate the position of the wild-type (13.8 kb) and the recombinant (15.4 kb) band. FIG. 13C shows PCR products of genomic DNA from the parental and a targeted clone amplified with the StopF2 and 5578R primers. Arrows indicate the position and the size of the expected products. The marker (M) and the sample lanes are not consecutive on the gel. FIG. 13D shows PCR products of genomic DNA from the parental and a targeted clone transfected with a cre-recombinase expressing plasmid and amplified with the Intr1-2 and 5578R primers. The arrow indicates the position and the size of the expected product after cre-mediated recombination. The marker (M) and the sample lanes are not consecutive on the gel.

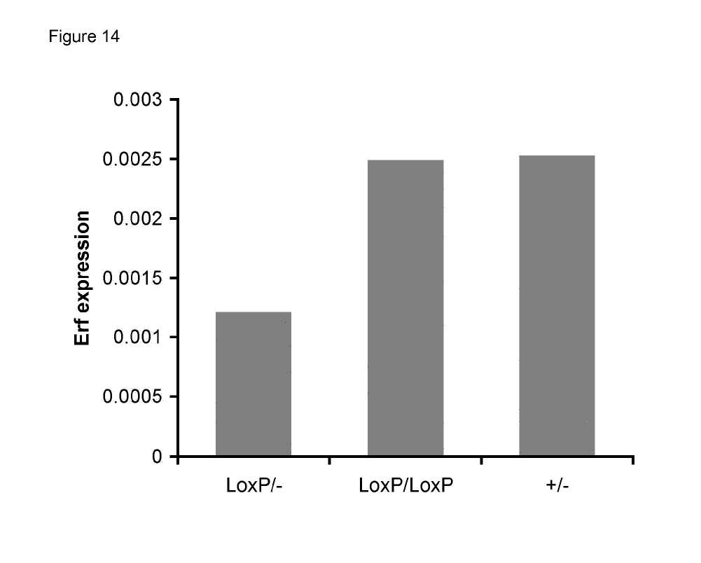

FIG. 14 shows Erf mRNA levels relative to GAPDH in livers from adult (2-3 month old) mice determined by QPCR.

### EXAMPLE 1

Exome sequencing was used to analyze the DNA from a 7-year old boy with craniosynostosis affecting the metopic, sagittal and left coronal sutures. His 15-year old brother had multisuture synostosis and their mother exhibited exorbitism and midface hypoplasia but did not have documented craniosynostosis (FIG. 1a-c). After excluding previously described variants and genomic regions for which the brothers did not share the maternal allele, 135 nonsynonymous sequence changes remained, including 5 nonsense mutations. One of the nonsense mutations (c.547C>T; p.Arg183Ter) was present in Erf, encoding an inhibitory ETS-family transcription factor located on chromosome 19q13.2. ERF was previously shown in a proteomics assay to be a prominent binding target of the paralogous kinases ERK1 and ERK2 (ERK1/2), key effectors of the RAS-MEK-ERK signal transduction cascade; the transcriptional activity of ERF is primarily regulated by ERK1/2-mediated phosphorylation, which leads to its export from the nucleus. Segregation of the mutation was confirmed from the maternal grandmother to the two affected children (Family 1, FIG. 4).

To analyze the possible role of Erf mutation in the phenotype, the gene was sequenced in a further 411 samples from unrelated subjects with craniosynostosis (Table 1) and 288 north European controls. Heterozygous mutations suggestive of loss of function were present in a further 11 patient samples but in no normal controls (P=0.004, Fisher's exact test) (FIG. 2 and Table 2). Multiplex ligation-dependent probe amplification was performed in 276 of the mutation-negative samples, but did not identify any additional deletions. Where possible earlier generations were analysed for the presence of the mutation, finding 26 mutation-positive individuals in total (FIG. 4). In 4 families the mutation had arisen de novo from either a parent (n=2), grandparent (n=1) or great-grandparent (n=1); in the 2 informative cases, the mutation was of paternal origin (FIG. 5). The occurrence of mutations only in patient samples and the identification of multiple de novo cases, establish that Erf mutations are the cause of craniosynostosis in these families.

The Erf gene encodes a ubiquitously expressed member of the ETS transcription factor family (which numbers 28 members in humans)¹¹, and acts as a negative regulator either by competing with other ETS-family members for DNA binding, or through unique targets. Functionally characterised motifs in the ERF protein comprise the N-terminal DNA-binding (ETS), central ERK1/2 interaction, and C-terminal repressor domains (FIG. 2b); DNA binding targets a core (5′-GGA^{A}/_{T}-3′) motif, with little sequence discrimination from other ETS family members. The mutations found are diverse; the 3 missense changes (1 recurrent) are located either in the initiation codon or in critical residues in the DNA-binding ETS domain, whereas the remaining 8 mutations comprise a splice site mutation, two nonsense changes and 2 frameshifts (1 recurrent, present in 3 families) (FIG. 2, Table 2, FIG. 5). The apparently synonymous c.21 A>T mutation, demonstrated to be present at the −2 position of a donor splice site, completely abolished normal splicing (FIG. 6a). Immunoblotting of cultured fibroblasts or lymphoblastoid cells from patients demonstrated reduced ERF expression associated with the initiation codon and nonsense mutations, but not the missense mutations affecting the ETS domain (FIG. 6b). DNA-binding domain mutants failed to repress Ets binding site-containing promoters (FIG. 6c). Collectively these data suggest indicate that the predominant pathophysiological mechanism is heterozygous loss of function (haploinsufficiency). The phenotype associated with this new syndrome was investigated in the 26 mutation-positive individuals (Table 3). Of 14 pediatric cases, 13 had craniosynostosis; in the 8 with accurate assessment by 3-dimensional computed tomography of the skull, fusion affected the sagittal (n=7), lambdoid (n=5), coronal (n=3) and metopic (n=1) sutures (FIG. 1h, Table 4), a pattern distinct from other monogenic types of craniosynostosis in which the coronal suture is most commonly affected. 7 of 12 probands had syndromic multisuture synostosis (Table 1), representing a 13-fold enrichment compared to other diagnostic groups (P=3×10″⁵, Fisher's exact test), but 3 subjects presented with single synostosis of the sagittal (n=2) or lambdoid (n=1) sutures only (FIG. 1f,g). In half of the families a diagnosis of Crouzon syndrome had previously been suggested because at least one individual had exorbitism and midface hypoplasia (FIG. 1b-e, FIG. 7, Table 3); however, FGFR2 genetic testing was normal. Chiari type I malformations (descent of cerebellar tonsils below the foramen magnum) were diagnosed in 4 cases (FIG. 1i); pathologically raised intracranial pressure was documented in 6 cases by direct monitoring, and diagnosed in 3 others based on skull radiology. Twelve (86%) pediatric cases had behavioural or learning problems, particularly affecting concentration and language acquisition; in three, the deficit in verbal IQ compared with non-verbal IQ exceeded 25 points on formal testing (see Table 3 for details). Notably, despite the multisuture involvement many affected individuals presented later in childhood than usually occurs in craniosynostosis, and primary surgery was frequently delayed (Table 3, FIG. 8). The remainder of skeletal growth was normal except for mild shortening of the digits in some cases, and no health problems of later onset were consistently found in carrier adults, in many of whom mild craniofacial signs or macrocephaly were the only clinical features. No genotype-phenotype correlation was evident. In 6 families, including 14 ERF mutation carriers, we used 3-dimensional scanning to document the facial phenotype; this showed that hypertelorism, shortening and/or vertical displacement of the nose, prominent orbits and forehead were consistently present but varied in severity (some mildly affected subjects previously had a clinical diagnosis of non-syndromic synostosis) (FIG. 1j, FIG. 9). This newly recognised disorder, which is termed ERF-related craniosynostosis, was identified in 5/402 (1.2%) of all patients requiring surgery for craniosynostosis in an extended Oxford cohort (children born between 1998 and 2006).

A specific role for ERF was not previously suspected either in the cranial sutures, or in osteogenesis more generally. In the mouse, heterozygous loss-of-function of the orthologous gene {Erf^{+/-}) is not associated with any abnormal phenotype, whereas homozygous loss {Erf^{+/-}) causes severe placental defects resulting in death by embryonic day (E) 10.5. To explore further the function of Erf during development, mice harbouring a conditional allele (Erf^{loxP}) containing a selectable marker, PGK-neo, located within intron 1, together with tandem LoxP sites to enable Cre-mediated excision were engineered (FIG. 10 a). Both heterozygous (Erf^{loxP/+)} and homozygous (Erf^{loxP/loxP}) conditional mice were grossly normal, but compound conditional/null heterozygotes (Erf^{loxP/-}) exhibited domed heads that became apparent during the first 3-6 weeks of life. Micro-computed tomography (CT) scanning showed that this was caused by craniosynostosis affecting multiple calvarial sutures (FIG. 3a-e, FIG. 10b). In addition the Erf^{loxP/-} mice were on average −18% lighter than their littermates, but no other specific skeletal abnormalities were evident on Alizarin red/Alcian blue staining at a variety of ages (FIG. 10c). Real-time reverse transcriptase-PCR analysis of ErfcDNA in dissected E16.5 mouse calvaria showed that in Erf^{loxP/30} compound heterozygotes, Erf transcription was reduced to 29% compared to the wild type (wt) (FIG. 3f). As in humans, the cranial sutures appear particularly sensitive to reduced Erf dosage, but the threshold level required for phenotypic manifestation is lower in the case of mice.

To gain insight into the developmental origins of craniosynostosis in the mice, Erf expression and calvarial osteogenesis in E16.5 mouse calvariae were examined by whole mount RNA in situ hybridization and real-time RT-PCR. In the wt, Erf is expressed along the osteogenic margins of the developing calvarial bones in a distribution similar to that of the master osteogenic regulator Runx2, haploinsufficiency of which causes cleidocranial dysplasia associated with defective calvarial ossification (FIG. 3g). Comparison between wt and Erf^{loxP/-} mutants for transcripts of Spp1 (osteopontin) or Bglapl (osteocalcin), markers of late osteogenic differentiation showed similar sutural gaps (FIG. 11a). However quantitation of transcripts in E16.5 calvariae showed modest (up to 2-fold) downregulation of multiple osteogenic markers in Erf^{loxP/-} mutants compared to wt littermates, significantly so (P<0.05; t-test) in the case of Prkg2 and Serinc5 (FIG. 11). At this stage therefore, ossification appears mildly delayed in Erf^{loxP/-} embryos. Hence further detailed analysis of postnatal stages will be necessary to dissect out the relative contributions of altered proliferation, differentiation, and apoptosis to the onset of craniosynostosis in the Erf^{loxP/-} mutants.

To gain insight into the possible nuclear targets of Erf, chromatin immunoprecipitation (ChIP) was employed in mouse embryonic fibroblasts using a previously characterized antibody specific to the C-terminal domain, combined with high throughput sequencing (ChIP-Seq). By comparing the enriched sequences from fibroblasts maintained without fetal calf serum for 4 hr (“−FCS”; Erk1/2 inactive; Erf nuclear), to those from cells grown in FCS (“+FCS”; leading to phosphorylation of Erk1/2, nuclear entry, and consequent phosphorylation and nuclear export of Erf), the component of the ChIP-Seq signal attributable to dynamic Erf binding could be identified (defined as a −FCS/+FCS ratio >3). Signals of dynamic binding were divided according to whether they occurred within 1 kb of transcription start sites (TSS; putative promoters) or at greater distances (non-TSS; putative enhancers) (FIG. 3h). MEME analysis identified two major sequences enriched near TSS (FIG. 12a), one corresponding to the ETS binding consensus and the other to the sequence bound by Ronin/Hcf-1; these motifs are virtually identical to those previously described in promoters bound by ETS1. In non-TSS, which are believed to provide a better indication of tissue-specific interactions of ETS factors, the three most highly represented specific sequence motifs were 5′-TGANTCA, 5′-TGTGG and 5′-TTCCT, corresponding to consensus motifs for API, RUNX and ETS factors respectively (FIG. 3h and FIG. 12a). This suggests that Erf binding sites frequently lie close to sites for other transcription factors; corroborating this, enrichment of both API and RUNX sites was observed in previous ChIP-Seq studies of other ETS proteins. The non-randomness of these associations was confirmed by demonstrating that closely adjacent API-ETS and RUNX-ETS sites both exhibit polarity consistent with interactions between pairs of transcription factors when binding DNA in specific orientation and separation (FIG. 12b,c). These data suggest that Erf regulates osteogenesis by altering the balance of positive and negative regulatory co-complexes formed by other Ets proteins (such as Ets2), Runx2 and AP-1 factors.

Previous data have implicated Erk1/2 activation in the pathogenesis of craniosynostosis caused by gain-of-function FGFR2 mutations in Apert and Crouzon syndromes. Consistent with these observations, genetic reduction in the level of Erk1/2 led to calvarial defects, but this was not associated with reduced expression of Runx2. Instead Erk1/2 has been shown to act post-transcriptionally by direct phosphorylation of Runx2, leading to activation and/or stabilization of the protein. Runx2 and Erf both exhibit particular dosage sensitivity in development of the calvarial bones, but acting in opposite directions; Runx2/RUNX2 deficiency causes reduced ossification (and RUNX2 excess has been associated with craniosynostosis), whereas here we show that Erf deficiency causes craniosynostosis. The RNA expression (FIG. 3g) and ChIP-Seq (FIG. 3h) data support a functional link between Erf and Runx2, with Erf potentially modulating Runx2 activity at both transcriptional and post-transcriptional levels. However based on the observation of modestly retarded calvarial osteogenesis at embryonic stages, the details of this mechanism are likely to be time- and tissue-specific.

In summary, the genetic observations in both humans and mice will focus renewed attention on the role of Ets factors in regulating osteogenesis, which although documented, has not been well defined. In addition the data provide a pathway-based phenotypic link with FGFR2 mutations, since several patients with ERF-related craniosynostosis were previously diagnosed with Crouzon syndrome (Table 3), and identify ERF as a novel target for therapeutic modulation of premature suture ossification.

### Materials and Methods

### Patients.

The clinical study was approved by Oxfordshire Research Ethics Committee B (reference CO2.143), and Riverside Research Ethics Committee (reference 09/H0706/20). Written informed consent to obtain samples for genetics research was obtained from each child's parent or guardian. In most probands the clinical diagnosis of craniosynostosis had usually been confirmed by computed tomographic scanning, although some individuals had skull radiography only. Venous blood was obtained for DNA extraction and preparation of lymphoblastoid cell lines. Fibroblast cultures were established from skin biopsies obtained from the scalp incision at the time of surgical intervention.

Exome Sequencing.

We used an Agilent SureSelect Human All Exon Kit (v. 1; 38 Mb) to capture exonic DNA from a library prepared from 3 ug of the proband's DNA (extracted from whole blood). The enriched DNA was sequenced on an Illumina GAIIx platform with 51 bp paired-end reads. 3.1 Gb of sequence was generated that, after mapping with Bowtie software to the Hg19 genome and removal of artefacts, resulted in an average coverage of 43 fold. Variants were called using the Samtools program (Table 5)

Segregation Analysis.

DNA from the proband, his brother and parents was analyzed using Illumina HumanCytoSNP-12 Beadarray (300 k). Chromosomal regions where the two affected boys shared the same maternal allele were identified and used to filter the Exome data (Table 5).

Mutation screening.

All cDNA numbering of ERF follows NCBI reference NM_{—}006494.2, starting with A of the ATG initiation codon (=1). The genomic reference sequence is available from NC_{—}000019.9. Mutation analysis was performed by direct sequencing of genomic PCR amplification products using the BigDye Terminator v3.1 cycle sequencer system (Applied Biosystems). Copy number variation was analyzed by multiplex ligation-dependent probe amplification (MLPA). RNA was extracted from whole blood taken in PAXgene tubes (Qiagen), and lymphoblastoid cell lines harvested in Trizol (Invitrogen), and cDNA synthesis carried out using the RevertAid first strand cDNA kit (Thermo). Primers for genomic and cDNA amplification, as well as MLPA, and all experimental conditions are provided in Table 6.

Three-Dimensional Facial Imaging.

Images were captured with a commercial photogrammetric device and manually landmarked, as were an additional 381 images of healthy controls used for comparison. Dense surface model and signature analyses were undertaken as described. Face signatures were visualized as colour coded heat maps, derived from lateral, vertical and depth differences of 24,000 surface points compared to corresponding positions on the mean face of the matched controls.

Western Blots.

Protein immunoblots were performed as previously described. Cells or homogenized embryos were lysed in RIPA buffer supplemented with protein and phosphatase inhibitors and equal amounts of protein samples were separated by discontinuous SDS-electrophoresis and transferred onto nitrocellulose. ERF was detected by the S17S anti-ERF rabbit polyclonal antibody (1:1000) and ERK1/2 by an anti-ERK1/2 rabbit polyclonal antibody (Cell Signalling #9102) at 1:1000 dilution in TBS with 0.1% Tween. Proteins were detected with an anti-rabbit horseradish peroxidase antibody (Jackson lmmunoresearch) at 1:5000 and visualized by chemiluminescence. Autoradiographs were quantified using NIH Image software.

Promoter Assays.

The mutations identified in patients were introduced into the wild type (wt) Erf cDNA by site-directed muatagenesis using the QuickChange™ mutagenesis kit (Stratagene) according to the manufacturer's protocol. Mutations were transferred both into the pSG5-ERF and pSG424-ERF expression vectors. The presence of the mutations and the absence of any additional changes was verified by sequencing. The ability of wt and mutated ERF to repress transcription activity was determined after transfection into HeLa cells and reporter assays. The pGL333 reporter carrying 3 copies of the GATA1 ets-binding-site (ebs) and the minimal TK1 promoter was generated by transferring the corresponding promoter fragment from pBLCAT333 vector to the pGL3-basic vector (Promega) and was used to determine repression on ebs-containing promoters. The pGLGal4 reporter carrying a gal4 DNA binding site and the SV40 promoter was generated by transferring the corresponding promoter fragment from the SV40/GAL4 plasmid to the pGL3-basic vector (Promega) and was used to assess ebs-independent repression.

Generation of conditional Erf^{loxP} mice.

All mice were maintained in a specific-pathogen-free animal facility at the Institute of Molecular Biology and Biotechnology, Crete or Biomedical Services Unit, Oxford, UK. Protocols involving mice were approved through the General Directorate of Veterinary Services, Region of Crete or the Oxford University Local Ethical Review process. Experimental procedures were performed in accordance with the European Union DIRECTIVE 2010/63/EU and/or the UK Animals (Scientific Procedures) Act, 1986 (Project License 30/2660).

The Erf targeting vector (FIG. 10a) was prepared by inserting a loxP sequence at the ApaI site 16 by 3′ of the Erf stop codon and a PGKneo-loxP cassette at the BstZ17I site 350 by 5′ of Erf exon 2, within the 7.3 kb Erfgenomic fragment 59A. The loxP site orientation was verified by sequencing and the targeting fragment was inserted into the pBSTK9 vector. RI ES cells were electroporated and selected as previously described and clones were screened by Southern blotting using a 2.2 kb BamHI-XbaI fragment (int-probe), for homologous recombination and absence of additional insertions. Positive clones were further tested for the presence of the 3′ loxP site by PCR amplification using the StopF2 (5′-ACCGAGATTCCTGAGAGCTAT-3′ SEQ ID NO. 9) and 5578R (5′-AGAGACTAAAGAGAGCTGTCC-3′ SEQ ID NO: 10) primers. Recombination after transfection with a Cre recombinase-expressing plasmid was tested by PCR using the Intrl-2 (5′-ATCATACATGTTTCTGAGGGG-3′ SEQ ID NO:11) and 5578R primers (FIG. 10a).

Chimeric mice were generated by microinjection of ES clones as previously described. Cells from clone no. 89 were injected into 3.5 days post conception C57BL/6 blastocysts and implanted into pseudopregnant CDI foster females. Male offspring with high levels of chimerism were mated to CBA×C57BL/6 females to produce mice heterozygous for the Erf^{loxP} mutant allele.

Skeletal Preparations.

Skeletons were harvested, fixed in 95% ethanol and stained with Alcian blue (0.03% w/v in 95% ethanol with 20% acetic acid) overnight. After several washes with 95% ethanol, the skeletons were rehydrated, treated with 2% KOH for 12 h and then stained in 1% KOH containing 75 ug/ml Alizarin red S for 24 h. Excess stain was removed by clearing in 1% KOH/20% glycerol and, after washing in 0.2% KOH/20% glycerol, skeletons were stored in 50% glycerol.

MicroCT Analyses.

Specimens for microCT were scanned using a General Electric Locus SP microCT scanner (GE Healthcare). The specimens were immobilized using cotton gauze and scanned to produce 14-28 um voxel size volumes. The specimens were characterized further by making three-dimensional isosurfaces, generated and measured using Microview software (GE).

Whole Mount RNA In Situ Hybridization.

Embryos were Dissected and Fixed in 4% paraformaldehyde, then dehydrated. In situ hybridization was performed as described using digoxigenin-incorporated riboprobes. The fir/probe was amplified from mouse cDNA with the following primers, mErf F2 5′-GCTGGAGAGAAGGCTCTAGGAGGCACTG-3′ SEQ ID NO: 12 and mErf RI 5′-GGTTAAGGCAGCAAAAGCTCAGGGAGTGG-3′ SEQ ID NO: 13 generating a 554 bp product which was cloned into pGem-T Easy (Promega). The Spp1 and Runx2 probes were kind gifts from John Heath and Georg Schwabe, respectively. For antibody detection, slides were incubated with antidigoxigenin antibody conjugated with alkaline phosphatase (diluted 1:1000, containing 2% fetal calf serum). Expression patterns were visualized using the BM Purple detection reagent kit (Roche). Whole mounts were analyzed using a Leica MZFLIII microscope and LASAF software (Leica Microsystems, Milton Keynes, UK).

ChIp-Seq.

For Chromatin Immunoprecipitation (Chip), 20-25 ×IO Mouse Embryo fibroblasts from E13.5 wild type embryos were grown in DMEM either supplemented with 10% fetal calf serum (+FCS), or in the absence of (−FCS) for 4 hours to induce Erf nuclear localization. ChIP was performed as previously described with the S17S anti-ERF rabbit polyclonal antibody. Briefly cells were fixed with 1% formaldehyde in PBS for 10 min at room temperature, nuclei were isolated and sonicated 50 mM Hepes pH 7.9, 140 mM NaCl, 1 mM EDTA, 1% Triton X-100, 0.1% sodium deoxycholate and 1% SDS and antibody was added over night at 4° C. followed by 2 hours incubation agarose-coupled protein G. The immunoprecipitated material was washed, de-cross-linked overnight at 65° C. and the DNA was purified by phenol extraction and ethanol precipitation. ChIP sequencing (ChIP-Seq) libraries were prepared and sequenced using the standard Illumina protocol.

Chip-Seq Analysis.

Paired-end reads from ChIP-Seq and input samples were aligned to the mouse genome build using Bowtie (version 0.12.3, http://bowtie-bio.sourceforge.net/index.shtml). Peaks were called with the SeqMonk program (version 0.19), using the contig generator function (Peak merge distance 50 bp, minimum peak size 50 bp, minimum fold enrichment relative to input was 5-fold). The number of reads in a union set of peaks from the −FCS and +FCS samples were quantified and normalized for total aligned read count in each ChIP-Seq and input sample using the SeqMonk quantification function. The in-house PERL script Smonker.pl was used to normalize the peaks against input and to calculate the difference in enrichment for each peak in the −FCS and +FCS samples and to store these values in a GFF3 file. Peaks were annotated for overlap (within 1 kb) with transcription start site (TSS UCSC Known Gene MM9 build) and problematic copy number regions of the MM9 genome (ploidy peaks) using the in-house PERL script intersectandappend.pl. The resulting annotated and quantified peaks were stored in a Multi-image Genome browser (MIG) SQL database (S. McG., in preparation). Peaks were filtered in MIG on the basis of the calculated relative enrichments between the two samples and their overlap with TSSs to produce the two datasets (TSS and non-TSS). Peaks associated with ploidy regions were excluded from the analysis.

Motif Analysis.

The de novo identification of over-represented motifs in a 300 bp region around the centre of each peak was performed using the MEME-chip suite of tools. The frequency of the identified motifs in enriched peaks and control peaks was calculated using the in-house program MotifQuant.pl. DNasel hypersensitive regions from adult fibroblast cells (from the encode project via UCSC table browser, file name: wgEncodeUwDnaseFibroblastC57b16MAdult8wksPkRepl) that overlap with TSS or not, as appropriate, were used as control regions. MotifQuant.pl randomly sampled from these control sets the same number of peaks as in the test set and repeated this sampling 1000 times to produce a mean frequency and a normal distribution for motif occurrence. For the motif analyses presented in Supplementary Tables 3 and 4 and FIGS. 12b and c, ETS binding motifs were defined by the sequences 5′ GGAA or 5′-GGAT: API as 5′-TCANTGA and RUNX as 5′-TGTGG.

TABLE 1
Patients with craniosynostosis analyzed for ERF mutations by DNA
sequencing

| | Non- | | | | | |
|---|---|---|---|---|---|---|
| | syndromic | | Syndromic | | Combined | |
| | | ERF | | ERF | | ERF |
| | | mutation | | mutation | | mutation |
| | Total | positive | Total | positive | Total | positive |
| Metopic | 46 | 0 | 13 | 0 | 59 | 0 |
| Sagittal | 70 | 1 | 16 | 1 | 86 | 2 |
| Unicoronal | 99 | 0 | 16 | 0 | 115 | 0 |
| Bicoronal | 25 | 0 | 24 | 0 | 49 | 0 |
| Uni- or | 13 | 1 | 0 | 0 | 13 | 1 |
| bilambdoid | | | | | | |
| Multisuture | 26 | 1 | 40 | 7 | 66 | 8 |
| Sutures not | 6 | 0 | 18 | 1 | 24 | 1 |
| specified | | | | | | |
| Combined | 285 | 3 | 127 | 9 | 412 | 12 |

TABLE 2
Mutations of ERF in present in 12 families

| | | Individuals | | | |
|---|---|---|---|---|---|
| Family | Proband | with mutations | | Predicted | De novo |
| # | ID | (see FIG. 4) | cDNA change | amino acid change | mutation |
| 1 | 0X2158 | II-2, III-3, IV-1, IV-2 | c.547C > T | p.Argl83* | |
| 2 | 0X2729 | II-1 | c.l512delT | p.Phe504Leufs*27 | |
| 3 | 0X2789 | II-1 | c.891_892delAG | p.Gly299Argfs*9 | |
| 4 | 0X3247 | 11-2,111-1 | c.256C > T | p.Arg86Cys | Y |
| 5 | 0X3248 | II-1, III-1 | c.194G > A | p.Arg65Gln | |
| 6 | 0X3 801 | 11-1,111-1, | c.3G > A | p.Metllle | Y |
| | | 111-2, IV-2 | | | |
| 7 | 0X3 970 | 11-2, 111-1, | c.891_892delAG | p.Gly299Argfs*9 | |
| | | 111-2 | | | |
| 8 | OX4097 | II-1 | c.891_892delAG | p.Gly299Argfs*9 | Y |
| 9 | 0X4626 | 11-2, 111-1, | C.1270OT | p.Gln424* | |
| | | 111-2 | | | |
| 10 | OX4708 | II-1 | c.256C > T | p.Arg86Cys | Y |
| 11 | OX4902 | 11-2, 11-3, | c.21A > T | p.Gly8_Phe9insl47 | |
| | | 111-1 | | | |
| 12 | OX5072 | II-1 | c.l201_1202delAA | p.Lys401Glufs*10 | |

1. A non-human transgenic animal comprising a modification in its genome which results in the animal exhibiting a level of Erf expression and/or ERF activity which is reduced compared to a wildtype animal, wherein said animal has a defect in ossification.

2. The non-human transgenic animal of claim 1 wherein said animal exhibits a level of Erf expression and/or ERF activity which is less than 50% of the Erf expression level and/or ERF activity of a wildtype animal.

3. The non-human transgenic animal of claim 1 wherein said animal exhibits a level of Erf expression and/or ERF activity which is from 5-49% of the level of Erf expression and/or ERF activity of a wildtype animal.

4. The non-human transgenic animal of claim 1 wherein said modification comprises a modification to the Erf gene.

5. The non-human transgenic animal of claim 4 wherein both alleles of the Erf gene are modified.

6. The non-human transgenic animal of claim 5 wherein one Erf allele comprises a null mutation and the other allele comprises a different modification.

7. The non-human transgenic animal of claim 4, wherein at least one Erf allele comprises at least one nucleotide insertion.

8. The non-human transgenic animal of claim 7 wherein said at least one insertion is made in an intron.

9. The non-human animal of claim 7 wherein the at least one insertion comprises an expression cassette comprising a promoter, marker gene and a first site specific recombination sequence and/or a second site specific recombination sequence

10. The non-human animal of claim 7 wherein the expression cassette insertion is present in intron 1 and the second site specific recombination sequence is present downstream of the Erf stop codon.

11. The non-human animal of claim 1 wherein said animal comprises one null Erf allele and one Erf allele comprising the insertion of claim 9.

12. The non-human animal of claim 9 wherein said first and second site specific recombination sequences are LoxP sites.

13. The non-human animal of claim 9 wherein said expression cassette comprises a PGK promoter and a neomycin resistance gene.

14. The non-human animal of claim 9 wherein said expression cassette is present in intron 1 of one Erf allele 350 base pairs 5′ of exon 2 and wherein said second site specific recombination sequence is present 16 base pairs 3′ of the Erf stop codon.

15. A non-human transgenic animal comprising an insertion of a promoter-marker gene cassette in one or both Erf alleles wherein the cassette is present in an intron of Erf and wherein said animal exhibits an expression level of Erf and/or ERF activity which is reduced compared to a wildtype animal.

16. The non-human transgenic animal of claim 15 wherein said both Erf alleles comprise the insertion or wherein one Erf allele comprises the insertion and the other Erf allele comprises a null mutation.

17. The non-human transgenic animal of claim 15 wherein said expression cassette comprises a first site specific recombination site and the insertion further comprises an insertion of a second site specific recombination site.

18. The non-human transgenic animal of claim 15 wherein said one or both Erf alleles comprises the insertion of claim 10.

19. The non-human transgenic animal of claim 15 wherein said animal exhibits a level of Erf expression and/or ERF activity which is less than 50% of the level of Erf expression and/or ERF activity of a wildtype animal.

20. The non-human transgenic animal of claim 1 wherein said animal is a mouse.

21. The non-human transgenic animal of claim 1 wherein said ossification defect is craniosynostosis.

22. Use of a non-human transgenic animal comprising a modification in its genome which results in the animal exhibiting a level of expression of Erf and/or ERF activity which is reduced compared to a wildtype animal in the production of a non-human transgenic animal which exhibits a level of expression of Erf and/or ERF activity which is reduced compared to a wildtype animal and which has an ossification defect.

23. Use of a non-human transgenic animal comprising a modification in its genome which results in the animal exhibiting a level of expression of Erf and/or ERF activity which is reduced compared to a wildtype animal as a disease model for an ossification defect.

24. The use of claim 23 wherein said animal is for the identification and/or screening of a treatment or test agent which increases Erf expression levels and/or ERF activity and which may be capable of treating an ossification defect associated with a reduced level of Erf expression and/or ERF activity.

25. Use of a non-human transgenic animal of claim 1 as a disease model for an ossification defect.

26. A cell line developed from a non-human transgenic animal of claim 1.


SEQUENCE LISTING (13 sequences)

SEQ ID NO: 1 - DNA, 3503 bp, Mus musculus
  source 1..3503: /mol_type=“DNA” /note=“Murine Erf mRNA sequence ” /organism=“Mus musculus”
attaacccgggaggcggcggcggggaggggagaggctctgagaggcgaggccgggtgcggcggcgagggcgtcccaacgggcgcgggacgggacggggcaacgcgggcgccaggagcagcggcccggagttggggcgccttgccccgggccccccagcatgaagaccccggcggacacagggtttgccttcccagattgggcctacaaaccggagtcatcccctggctccaggcagatccagctgtggcactttatcctggagctgcttcggaaagaggagtaccagggcgtcatcgcttggcagggggactacggggagtttgtcatcaaggaccctgatgaagtggctcgcctctggggggtccgcaagtgcaaaccccagatgaactatgacaagctgagccgggctttgcgctattattacaacaagcgcattctacacaagaccaaggggaaacggttcacctacaagttcaacttcaacaaactggtgctggtcaattaccctttcatcgatatggggctggctgggggtgcagttccccaaagcgccccaccagtgccatcaggcggcagccatttccgcttccctccctcaacaccctctgaggtgctgtcccccactgaggatccccgatctccaccggcttgttcttcatcatcctcttctctcttctctgctgtggttgcccgacgcctgggccgaggctcagtcagtgactgtagtgatggcacctcagagctggaggagcctctgggagaggaccccagggcacgaccacctggccctccggagctgggtgccttccgagggccccccctggcccgcctcccgcatgaccctggtgtcttccgtgtctatcctcggccccggggtggtcctgaacccctgagtcccttccctgtgtcacctttggctgggcctggctcccttctaccccctcagctctccccagctctgcccatgactcccacccacctggcctacacaccctcacccacgctgagtcctatgtaccccagtggtggtgggggccctagtggctcagggggaggttcccacttctccttcagtcctgaggacatgaaacggtacctgcaggcccacacccaaagcgtctacaactaccacctcagtccccgcgccttcctgcactacccagggctggtggtgccccagcctcagcgccctgacaagtgcccactgccgcccatggcaccggagaccccgccggtcccctcctcagcctcgtcttcctcttcctcctcttcatccccgttcaagtttaagctgcagccacccccgctaggacgccggcagcgggcagctggagagaaggctctaggaggcactgacaagagcagtggtggcagtggctcgggtggactggctgagggggcaggtgcagtagctcccccaccgccaccaccccagattaaggtggagcccatctcagaaggagagtcggaggaggtggaggtgactgacatcagtgacgaagatgaggaagatggggaggtgttcaagactccccgtgccccgcctgcaccccccaagccagagcccggagaggcaccgggggtggcccagtgcatgccccttaaactgcgctttaagcggcgctggagtgaagactgtcgcctggaggggggcgggtgcctgtctgggggccctgaagatgagggtgaggacaagaaggtgcgtggggacgtgggccctggggagtctgggggaccccttaccccacgacgggtgagctctgacctccagcacgccacagcccaactctccctggagcaccgagattcctgagagctgtgggcacgggcccacccccacccacccaccaccccactccctgagcttttgctgccttaaccgccccgtccccgcctcccccccccccccccccccagtcccggaggtgaggacagctctctttagtctcttccctgcctcctccctttctcctcccccacattttgtataaaactgaattttttttttaatggtagggtgggtgggtgcccagagctgggggctaatctgttcctcatctgggtttctaagttctgggcaaagtggtggtggggggtgggaggggggtgttaagggggggcacctccattctgggaatttctatttgaacagaggctttggccttcaaacccaggaacttttttattacaatcgtaggaagaaaagccttgtccccctccctcttctctgcctcttaccttgtcctcccccaccccaatccatgcccctcccccagccctaaccctgcctttcttgcccctccaggggctgtgagtgcctgggtttcttataccccacaaggttgcagcaggggcaggagggacaaattttataaaccaaaaattctgtggggggtggtggggtgggaggcagaggaaggtgaggggtgccgcctgtgggccacaaatctctacaagtgcctgttccctacaccacccagtactggtccagtcccttcacgcccaccccctgctgctcctaggtctggcccatgggcaggtgggtcaggggacaggaagggggggttgtctctcaaaaccaaactggaagcccctctctgcctcctagctggggccccaggggtggcctggagggctgcggtcaagccttattctgtattgggaatggagggtggggtgggacgtgagggggctgctggagaatgtattcaaaacaataaaactttggacctttgcatgtggtaatttatcctaggtgctgggcgcagccacagtatcaacagagacttggcttaattctggacaggtctagacacctgcttcccatctgtggccagaaggcctgactccacctgaacaggtcaactaggcctggggagttattggcctactttgagatgtcacaagcctcagagctgatgattaaggtttctggctttccgtagtcttgtctttccttgactggttccttatcatcccatctgcctctaggacccttaacaaggccctgctgttcctggttctcctacctgaattgcttttgccctccagctctgactgctaagccacacccccatcggttccccagggaaggaagggtcccgtattttgaatcctccccatcttactccaatgtactcggaggaggctttagtggtccacacctgtaatcccaccagcacttgaggtaggaagttcaaagttaaggccaggaccatgtgaaagggaggtgtgcgatgttagccttctgcttgggatcctttgaccgggaggcctgtagtgtcctgaagtgcagcccttgggctggagagacgctcggctctgtgttcaagggtgcttctgctcttcggagaacctccgtctgttgcagagtacctcatgtggggtggctcgtgacagctcacagcttcagttccaggggaggcagcttctggcaaccactcagaattggcatagagtcactcagatatatgcattaataaagatctttttaagaaattta

SEQ ID NO: 2 - protein, 551 aa, Mus musculus
  SOURCE 1..551: /mol_type=“protein” /note=“Murine ERF sequence ” /organism=“Mus musculus”
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDMGLAGGAVPQSAPPVPSGGSHFRFPPSTPSEVLSPTEDPRSPPACSSSSSSLFSAVVARRLGRGSVSDCSDGTSELEEPLGEDPRARPPGPPELGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKALGGTDKSSGGSGSGGLAEGAGAVAPPPPPPQIKVEPISEGESEEVEVTDISDEDEEDGEVFKTPRAPPAPPKPEPGEAPGVAQCMPLKLRFKRRWSEDCRLEGGGCLSGGPEDEGEDKKVRGDVGPGESGGPLTPRRVSSDLQHATAQLSLEHRDS

SEQ ID NO: 3 - protein, 548 aa, Homo sapiens
  SOURCE 1..548: /mol_type=“protein” /note=“Human ERF sequence ” /organism=“Homo sapiens”
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKAVAGADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 4 - DNA, 2705 bp, Homo sapiens
  source 1..2705: /mol_type=“DNA” /note=“Human Erf mRNA ” /organism=“Homo sapiens”
attaacccgggaggcggcggcggggaggggagaggctctgagaggcgaggccgggtgaggcggcgagggcggcccgacgggcgcgggacgggacggggcagcgagggcgccgggagccgcggcccggaatcggggcgcttcgccccgggccccccagcatgaagaccccggcggacacagggtttgccttcccggattgggcctacaagccagagtcgtcccctggctcaaggcagatccagctgtggcactttatcctggagctgctgcggaaggaggagtaccagggcgtcattgcctggcagggggactacggggaattcgtcatcaaagaccctgatgaggtggcccggctgtggggcgttcgcaagtgcaagccccagatgaattacgacaagctgagccgggccctgcgctattactataacaagcgcattctgcacaagaccaaggggaaacggttcacctacaagttcaatttcaacaaactggtgctggtcaattacccattcattgatgtggggttggctgggggtgcagtgccccagagtgccccgccagtgccgtcgggtggtagccacttccgcttccctccctcaacgccctccgaggtgctgtcccccaccgaggacccccgctcaccaccagcctgctcttcatcttcatcttccctcttctcggctgtggtggcccgccgcctgggccgaggctcagtcagtgactgtagtgatggcacgtcagagctggaggaaccgctgggagaggatccccgcgcccgaccacccggccctccggatctgggtgccttccgagggcccccgctggcccgcctgccccatgaccctggtgtcttccgagtctatccccggcctcggggtggccctgaacccctcagccccttccctgtgtcgcctctggccggtcctggatccctgctgccccctcagctctccccggctctgcccatgacgcccacccacctggcctacactccctcgcccacgctgagcccgatgtaccccagtggtggcggggggcccagcggctcagggggaggctcccacttctccttcagccctgaggacatgaaacggtacctgcaggcccacacccaaagcgtctacaactaccacctcagcccccgcgccttcctgcactaccctgggctggtggtgccccagccccagcgccctgacaagtgcccgctgccgcccatggcacccgagaccccaccggtcccctcctcggcctcgtcatcctcttcttcttcttcctccccattcaagtttaagctccagccgcccccactcggacgccggcagcgggcagctggggagaaggccgtagccggtgctgacaagagcggtggcagtgcaggcgggctggctgagggggcaggggcgctagccccaccgcccccgccaccacagatcaaggtggagcccatctcggaaggcgagtcggaggaggtagaggtgactgacatcagtgatgaggatgaggaagacggggaggtgttcaagacgccccgtgccccacctgcaccccctaagcctgagcccggcgaggcacccggggcatcccagtgcatgcccctcaagctacgctttaagcggcgctggagtgaagactgtcgcctcgaagggggtgggggccccgctgggggctttgaggatgagggtgaggacaagaaggtgcgtggggaggggcctggggaggctggggggcccctcaccccaaggcgggtgagctctgacctccagcatgccacggcccagctctccctggagcaccgagactcctgagggctgtgggcaggggacctgtgtgccccgcaccccccatgcttcttttgctgccttaagccccctatgccctggaggtgagggcagctctcttgtctcttccctgcctcctcccttttccctccccacattttgtataaaactttaatttcttttttttaaaaatggtgggggtgggtgggtgcccagggctaggggctattccctgtctctgtgggtttctaagctctgggcaaagtggtggtagggggagggagggggaagttaagggggtcacctccattctggggaatttatatttgaattgaggctttggccttaacacccaggaacttttctattacaatcgcttaggaagtaaagccttgtctccctccctgttctctgcctcttgtacccctctgacccacccgctctgccccactcccagccctcctcagccccagccctgcctgccctgcccctccagggggccatgagtgcctaggtttctcataccccacaaggtcacagcaggggagggagggacaattttataatgaaccaaaaattccatgtgttggggggtggggggcggaggagggtgaggggtgccgcccatgggccacaaatctctacaagtgcctgctatccctctcccactccccaccccagcaccggtccaaccccttcatccccagctgctcctaggactggcccatgggcaggcgggtggggggatgggaagggggtgccctgaaaccaaactggaagccccctctgcctcccagctggggcctctggggtggggtggggggctgtggtcaagccttattctgtattggggactgagggtggggggagtagaggggccgctggagaatgtattcaaaacaataaaactttggacctttggatgca

SEQ ID NO: 5 - protein, 34 aa, artificial sequences
  SOURCE 1..34: /mol_type=“protein” /note=“LOXP” /organism=“artificial sequences”
ATAACTTCGTATAGCATACATTATACGAAGTTAT

SEQ ID NO: 6 - DNA, 34 bp, artificial sequences
  source 1..34: /mol_type=“DNA” /note=“FRT” /organism=“artificial sequences”
gaagttcctattctctagaaagtataggaacttc

SEQ ID NO: 7 - DNA, 10199 bp, Mus musculus
  source 1..10199: /mol_type=“genomic DNA” /note=“Genomic sequence for mouse Erf gene (including flanking se quence)” /organism=“Mus musculus”
gagaatatagctctgcttcccctctacccagtagcaggctgcctacctgcatccgatcggtaccccgatttcctgtttcccgagcctcaggctccatccattcctcctatctgctgcagcgaacacagacttaacacttcccctacgctcgggcctttggtcttaagccctgtcttcctctctcctggcgacccccaatgctcctccggagcacatttgttcagacttgggtcgcccccagcatcctggcactatctccggaaccccctgctccagtgtggaggtgggggtacctccggccgcgttcccaggacagctgcagcctaagggacagtggtgattgtgggggctcccgaatgcattctggtcacttcctccccagctggactgggtctatcaggaaggccaagaccccatctccacaggagtcgagggtacagccgcctccccatttccgatgctgggaaatgtttcccacccattccaccccacccctggacagctgccctctttcaggctcggggttgagggggttgggtaaactcaggctgggaaatggctgtgctcttgatcatcctccaagatccagactccgcccccctgcggagaagactctgccccttccccacaaaaatttctgatcgtcccccttcatataattcctttcctccaaaaaggtcccaaaagaaactctggagccaggaaagggcagaagagaggcagcaggtactgagtgctgaagacttaagttcttctgacttagagcctccaagaagggtcgtgacccccaggacctccccagcccctccctgcccttcctccccccccccccccccccgccgcgcggcccctttaagccaagagccggccggtcctcagtggctgcgcgccggcaagcgtgtgtgagtgcgcgggagggggcgggcacagtgtctccatggcgacgcggcggtgacgtcgccggccggggggcgtgggcgtcccggccccggagtgcgatattaacccgggaggcggcggcggggaggggagaggctctgagaggcgaggccgggtgcggcggcgagggcgtcccaacgggcgcgggacgggacggggcaacgcgggcgccaggagcagcggcccggagttggggcgccttgccccgggccccccagcatgaagaccccggcggacacaggtgggggcgggggcagcacgtgtgggggagacttgttgccccgggaaaccgggagggaaaactttgggtgggggctgaaggggttctggggagcccgattgatacccagttcttggggacccccaagcactcgagacctgttgcttctagaggcgcgagctggggtgctgcctgggagccccaattctgtggatcctttcggaatggtatgggtgctgcgggtgctgggctttcccttcccggactcggcaacctgggttctcccgggatcactggggtggcagagtcccctccctgtcgttctgtcacgttgcactccgggggccgggacgtgtgcggtgcgaggtgggggttctgtacagggacccgctatgctcggttctgcgctgcgcggtccggcccgtgaccttcacggcagcgcgtgcccagcacccggtgagcgggatacgggatggcctggagctgggcgtgagaggggaacacgggactcggggtccgggctgcaccgcccctcccccgtccgtctcctcgctcccccgcccggactgagtcgcaaagttccaaagcgcagcccgaacgttcgaaaaaggaactttttgtttccgaccttagaacgcgcggctttaaggtggctcaggggagacttgggtaccctcctccggcgcgggcggaggctgggagttgaggagggtgggaaagatgggggggggggggggaatgcagaggctctcccttcgtccttccccctccccgatcctgcgcttctttcagcgggccgtcttttccctccgcccctctcttccccgccaagttgctcctccttaaggggccaggattcccctccccccccatcgccgggcggggggcagcaggattaaccccccctcccactcggactttcccccttcccctcccccgccgcccccgggctcgcacaggctggactttgcgcccgggcgcgcggggggagggggcctcgccgccccctgcccgtctgccccgtcggaatccggcccggggcggggacgaggggagggggaagggggccgggcgccgctgttcaaacttgtttccttcccccggcggcggccgcggctccggctcctatgtgcgggtgtgtgccccgaccggcccacccaccccacctccctgctcggcgggtgggggcggctcttaaagggctcggagcagggctccggcgccaggggattggggccaaggaagggggtcgggagtggggtaggggtgtgcagtgccatagggtcagtcacgtgcgtcccacttactccccactccccgactcccacaggctcgtgagctgccgggccctgggggcagcaggagcctcgcctcaccggggaggggaccaaggcgcccagacaaccccgccttgtcttccctctatgttacgggccttggcttgacggaacctgtagggccactgcaaccctgtctggggtcccagtcatgtgtagattctcaagggaaggcgagagaccagaacggcctcgaggtcggtctgtttctccgactgctggccactgctagcccttccatccgatcctccatcccggcgtttttctgctccgccgtggctgtttcccacttcctgttttcttcccggctctggcgccatcgaggtctgcctggagtgggggaggtgttgaggccccccgggaggcagatgtatttttcctcaaaccccccccccccaatctgacccctatattcccataaaaagtttaggcagtcatgttaagttggggattgtttcccagaacttatgtcccgacccaggcttgcacagtctgggatatccaggtgtgccttctgggacccagccatcatgtccttcctgtctgcagctacctctacaccctgtctgcgtttctggagcccttctctcagtgcagctgctttcttaaagcctcactggacccccatgctcgagaggtcttgcaccctcaagctttcttgctgcagagctggtgtttccgtgatttaagcacgtggacccgtatacaacacacacatggtctccttgcccgtgcctggctcctggctccaaggcctggggaacagagccccattgagacaccctgacgtcacccccactccgtcccctccccaaagctgggaacaggccctcgtgtatgctgtgtccgatgaggaaagccggtactagggtggtgctggggtgggcctgggcatgaggaggggtctctgccagaagtagaagccagggtccaggccctgggggtggtgtttcccaggaaggaggttagcaagggggcctgggacagctcccagaatgcgtgcttaggcctttccagagcccccccacctcctttccgagttccctttgtgttgcatgtagtctctctcactccctctcttctgcaaagtttatttttagcctcctgctcccccgctcccaggactgtcgctgcacacacgttgcctggttaccgggacacggaggctggggctggtgggctgggaagggtggctttaattttggggggagggtggaagtcagtacacccctccacctccggactgatgtcttaggggccttagcccccctcttgggacagggagccaggttggaactgagataagagagtctgtggagctttgtgccctgaagggagccaggagttttttttggaggtagttccacggtatatggaggggtgacttgatgacttgggttcccaaagacctcaggaccctaatggataatgcagggccttggttccctatagcaggacaaggatgctgggatgaacagctgggttctcaagagaaggatgaggctgtggatgctggggtccttagaaggggttgtcaggggctgtcaccgaggactgctgggccaggactctgggcccaacatcctacacagctggaagccctgtcctggaacctggcagtggctggctggtttgagatggggatttctgaatggggcatgtggactaaggagtgttgagcaggccatccctttctacctagcccagggttcgactgcctgcctgttcaggggaacactgggttatgcattgggaaaggtggtttctgaaggaggaaaaggaccacttttggatttcagattgggggttgaggcttcggggagactgaatctcgcacagagtcttgcttgctttctgtttgaattggaccccgcccatttttaaccatataccttggtcttccttctatagtggctcccgggcccctgatctgcctccccccttccgtgccttcctcccctttccctcctcccacccctccattaccggctgcctagtgctgaattattgatggcccctgattacccgggggtttgggaaatgacaacaaccgcagcccccccaccccaggctgcccaccccctccagggaccaagtcagcccgccaccaacctccacccaaggttaatgctgggggcgggccagcaggtgggctgagagttagccaaggatgagaaggcagaagaggcaagaagatgacagtcctgagcctgcctgggctgtctgggctgggttccctccatccaggtcctaagggtggacttgaggagggcattattggctcagagggtccagtgctgtgggggttgggggggctctcagacagtctgggtttcactctcaagggaagacagaagcctctgtgtgtgcaagcgctgacctcatcgtcaaccagccaccccacccggcctggtccttttaagaaaaaaaaaatactctccagtcctgggtgggagtaggagcttctataacctgagagaaagatcatacatgtttctgaggggagggccacagaagcagaggggcactgctagctctgaatggaagttggggtacacatagggggctgtgtccccatcctgctggccacgtgtacccttttctgcccctcaccctccctcactggcagccctgcccgccatcctggcttggtgcctggccccctggcactggccccgggacccgccgccgttggtttcctgtttctcccggtgtcgctggagccgactccagcttcccctcctcccagctcctgtctaggcccctcacgcagatgcccatacggttactctcagatttgggtcttaatctggctgaggaaaagggctttcctgatggtggaagactgaggggtggggcaggacataaaaactccagtaggagccagagcccccaccttggcatcttgaccccaggcaactccctgtctcctgtccctagggtttgccttcccagattgggcctacaaaccggagtcatcccctggctccaggcagatccagctgtggcactttatcctggagctgcttcggaaagaggagtaccagggcgtcatcgcttggcagggggactacggggagtttgtcatcaaggaccctgatgaagtggctcgcctctggggggtccgcaagtgcaaaccccagatgaactatgacaagctgagccgggctttgcggtgagaaggggctgtgggccccagaggacatgtggcaggtccctatggttggaaattcttgatacataaggacaggttttgggtttgatgtctagctggctctgctaggtctagctgtgtcaacttgggccttgggcaagtcctgccagtcttgtgaactcataggtcatacagaggtgtcccaggcaaagaaatgtagctggagactctagtgtgctcctggttacttgatttctcggctgttgggtaccctttatgcatgagacccagccttgtgtcatggtgcctgggtcctgtggagggtggcttgatggcccgatggtgttttatgcccacagctattattacaacaagcgcattctacacaagaccaaggggaaacggttcacctacaagttcaacttcaacaaactggtgctggtcaattaccctttcatcgatatggggctggctggtgagtgtgtggcctgcgtgtcagaagagggtgaagaggtggggttttctgtgttcagaggagaccagagaacatgatgcctactctcccttcttttgtcagggggtgcagttccccaaagcgccccaccagtgccatcaggcggcagccatttccgcttccctccctcaacaccctctgaggtgctgtcccccactgaggatccccgatctccaccggcttgttcttcatcatcctcttctctcttctctgctgtggttgcccgacgcctgggccgaggctcagtcagtgactgtagtgatggcacctcagagctggaggagcctctgggagaggaccccagggcacgaccacctggccctccggagctgggtgccttccgagggccccccctggcccgcctcccgcatgaccctggtgtcttccgtgtctatcctcggccccggggtggtcctgaacccctgagtcccttccctgtgtcacctttggctgggcctggctcccttctaccccctcagctctccccagctctgcccatgactcccacccacctggcctacacaccctcacccacgctgagtcctatgtaccccagtggtggtgggggccctagtggctcagggggaggttcccacttctccttcagtcctgaggacatgaaacggtacctgcaggcccacacccaaagcgtctacaactaccacctcagtccccgcgccttcctgcactacccagggctggtggtgccccagcctcagcgccctgacaagtgcccactgccgcccatggcaccggagaccccgccggtcccctcctcagcctcgtcttcctcttcctcctcttcatccccgttcaagtttaagctgcagccacccccgctaggacgccggcagcgggcagctggagagaaggctctaggaggcactgacaagagcagtggtggcagtggctcgggtggactggctgagggggcaggtgcagtagctcccccaccgccaccaccccagattaaggtggagcccatctcagaaggagagtcggaggaggtggaggtgactgacatcagtgacgaagatgaggaagatggggaggtgttcaagactccccgtgccccgcctgcaccccccaagccagagcccggagaggcaccgggggtggcccagtgcatgccccttaaactgcgctttaagcggcgctggagtgaagactgtcgcctggaggggggcgggtgcctgtctgggggccctgaagatgagggtgaggacaagaaggtgcgtggggacgtgggccctggggagtctgggggaccccttaccccacgacgggtgagctctgacctccagcacgccacagcccaactctccctggagcaccgagattcctgagagctgtgggcacgggcccacccccacccacccaccaccccactccctgagcttttgctgccttaaccgccccgtccccgcctcccccccccccccccccccagtcccggaggtgaggacagctctctttagtctcttccctgcctcctccctttctcctcccccacattttgtataaaactgaattttttttttaatggtagggtgggtgggtgcccagagctgggggctaatctgttcctcatctgggtttctaagttctgggcaaagtggtggtggggggtgggaggggggtgttaagggggggcacctccattctgggaatttctatttgaacagaggctttggccttcaaacccaggaacttttttattacaatcgtaggaagaaaagccttgtccccctccctcttctctgcctcttaccttgtcctcccccaccccaatccatgcccctcccccagccctaaccctgcctttcttgcccctccaggggctgtgagtgcctgggtttcttataccccacaaggttgcagcaggggcaggagggacaaattttataaaccaaaaattctgtggggggtggtggggtgggaggcagaggaaggtgaggggtgccgcctgtgggccacaaatctctacaagtgcctgttccctacaccacccagtactggtccagtcccttcacgcccaccccctgctgctcctaggtctggcccatgggcaggtgggtcaggggacaggaagggggggttgtctctcaaaaccaaactggaagcccctctctgcctcctagctggggccccaggggtggcctggagggctgcggtcaagccttattctgtattgggaatggagggtggggtgggacgtgagggggctgctggagaatgtattcaaaacaataaaactttggacctttgcatgtggtaatttatcctaggtgctgggcgcagccacagtatcaacagagacttggcttaattctggacaggtctagacacctgcttcccatctgtggccagaaggcctgactccacctgaacaggtcaactaggcctggggagttattggcctactttgagatgtcacaagcctcagagctgatgattaaggtttctggctttccgtagtcttgtctttccttgactggttccttatcatcccatctgcctctaggacccttaacaaggccctgctgttcctggttctcctacctgaattgcttttgccctccagctctgactgctaagccacacccccatcggttccccagggaaggaagggtcccgtattttgaatcctccccatcttactccaatgtactcggaggaggctttagtggtccacacctgtaatcccaccagcacttgaggtaggaagttcaaagttaaggccaggaccatgtgaaagggaggtgtgcgatgttagccttctgcttgggatcctttgaccgggaggcctgtagtgtcctgaagtgcagcccttgggctggagagacgctcggctctgtgttcaagggtgcttctgctcttcggagaacctccgtctgttgcagagtacctcatgtggggtggctcgtgacagctcacagcttcagttccaggggaggcagcttctggcaaccactcagaattggcatagagtcactcagatatatgcattaataaagatctttttaagaaatttacacagatgcatatgaatgtttggtctatatgcatgtccgtgcacctgcatccgagatggtgagaaaggggcgtcagatgctccggaaggacagaaagatagttgtgaactgccgtgtgggtgctagggaccgaccctaggtcatctgcaagagcagcaagctctcttaaccaccaagccgtcttgccagcccgaataaataaaactatttttttttttttttttggtttttcgagacccggtttctctgtatatccctggctggcctggagctcactttgtagaccaggctgacctcgaactcagagatctgcctgcctctgcctcctgagtgctgggattaaaggcgtgcgccaccactgctctgcttataaaaccaaatcttaaagaaaccttggaaagtaatcggtcatccttgtcccaccagctcacctcctgcacgcaccccccacccccaagcagattttaagagggatttgttgactgacctgacctgctgtgtgccaagtgtttaagcatttcagtttctcctttcctttttttttttttttttaatatttttttttctgggatggtttctctgtgtagccctggctgtcctggaacttctgttgagcaggctggcctccaactcagatccacctgcctctgcctcctgggattaaaaaccctgtgcactctcgaacacagacacacacacatcccagctcaggccccaccgcagcacattttagttctttaagcattcagcaacaaagggcttcattaaacttgcctgtagacctagaattgagctattcgacaaagtagcgcttaatctaatgtcttaacaacagctggattctccagtggaaagccagttggagtccagatcgagggatctggggtcttagaatttggttgatgaggagcctatagagctaagaaaccaggagctttcagtgctgagtctttgaggagataaaacaatcaggaagaggccgggctctgggtgctttagg

SEQ ID NO: 8 - DNA, 9593 bp, Homo sapiens
  source 1..9593: /mol_type=“genomic DNA” /note=“Genomic sequence for human Erf gene including flanking seq uence ” /organism=“Homo sapiens”
tttggcctctaccctgctgccggctcctgggctccatcagatctgcgctgtgatttcctgtttcccagagcctcaggcttcatccttccctcccggctgaggcagagaacggagacttaacacttctctacgctctggctttcgatcttaagccctgtcttcctctctcctggcgaccctcagtgccccttgggagcacatttgttcagactttggtcgccccctcgtatcccggccccgcctccggaacccccggtcctagtagtggacgtggggggacctccggcgccagccctatccccgggacagctgcagcctggggacggtggtggtgggggcttccaaatgcattctctctggtcacttcctccccagctggacgggggttatcagggaagccaagaccccatccccacaggagcggagggcacagccgcctccccatttccgatgctgggaaatgcatcccacccattccaccccacccctggacagctgcccccattcaggctcagggttggggggttgggtaaaaccaagctgggaaatgctgtggtcttcatcatcttctaaggtcctgaccccgcccctctgcagagaagattctgccccctccccacaaaagtttctgacccccgcttcctcggaagggccccccgttttcccatacaggctcccaaaagaaactctggagcccgcagtgggcaggagaggaggaggggcagcaggtagcgagtgccgaagacttaagttagctgcagactcggagcctctgactagggtcatgacccgcgggaccccatcccacccccaccccctccttcctccctcccccgccgcgcggcccctttaagcccagagccggccggtcctcagtggctgcgcgccgacgagcgtgtgtgtgagtgcgcggggagggggcgggcgcagtgtctccatggcgacgcggcggtgacgtcgccggccggggggcgtgggcgtcccggccccggagtgcgatattaacccgggaggcggcggcggggaggggagaggctctgagaggcgaggccgggtgaggcggcgagggcggcccgacgggcgcgggacgggacggggcagcgagggcgccgggagccgcggcccggaatcggggcgcttcgccccgggccccccagcatgaagaccccggcggacacaggtgggggcgggggcagcacgtgtgggggagacttgttgccccgggaaaccgaacggagaaactttgggggggctgaaggggtccgggggagcccgagtgatccccagttctgggggacccctcaagcactggagacctgttgcttctcgtggcgcgagcggggttgctgcctgggacccccgattcctggctccgggcggagtggtgtgggtgctgcgggtcttggtcctgtatccctaccgggaccccgcaggccgggctctcccccactcccgggatcacgggggtggcggggtccttcccctccgctcttgtcacgttgcactctgggggccgggacgtgtgcggggcgggggggctcgtccaccgggcccgctatccctggttctgcgctgcgcggtcctgctggtgaccttcacggcggcgcgtgccctgcaccaggttagcgggggatacgggatggcctctaggaggagggcgtgagtggggagcccgggactcggggtcctggctgcgccgcccctcccccgcccgcctctgtcggctcctcgctcccccgccctcgccgagtcgcaaagttccaaagcgcagcccgaacgttcgaaaaaggaactttttgtttccgaccttagaacgcgcggctttaaggtggctccggggagacctgggtaccctcctcccgcgccaccggtggcccggggttgaggagggtggggaaggtggggggagacgcagaggctcccccctcgccccccctcccggatccacctcttctctttcagcgggccgtctcttccctccgcccctctcttccccgccaacttgctcctccttaaggggccaaggttcccctccccctcggtcggcgggcggggggcagcgggattaactccccctcccactcggacttttccccctcccctcccccgccgcccccgggctcgcgcgggctggactttgcgcccgagcgcgcagggggaggggtcccagccgccccctgcccgtctaccccgtcggaatccggcccggggcggggacgggggagggggaagggggccgggcgccgctgttcaaacttgtttccttcccccggcggcggcggcggcggcggcggcggctcgggctcctgtgtgcgggtgtgtgccccgaccggcccacccaccccaccccatccccaccccattcccaccccattcccatccccatccccctgctcggcgggtgggggcggcttaatcttaaagggctccgagctgggatccggcgccaggggattgggacccggaccgaggaggggccctgggggtggggccggggtgcggagtgcggtagggtcagtcacgtgcgtcctacacactcccccggcttccactcgctcccgggtggctgggccctgggggcagcaggagcctaccctcgcccaaggaggaggccgaggcacccagacaacctcgcttcatcttctctccctgctaccggccttggcctgataggatccgcgggtcgcttccccaaccctgtcggggggcgggacagtgtgtgggttttccggctgggggaggggaggggaaggaggcctggaatggcctccgatcggtgcatctttcccgtctgcctcctggcctgccgctccgcacccctccatccagttctccatcccggtccctctctctgccgtggctgtttcccacttcctgttttcttcccggctctggcgccatagagggccgcctggagtgggggaggtggtggggccccccaggaggccggcgtgttcttcctctgccaccactcctcccttccgaccctcatagtcccttaaaaaaagtttgggccgtcacgtgaggttggggggtgcccaggagttttgtccccccaggcttgcatagtctgtctgggagaatccctaccccgcctctgtgggacccaggtgtgcttctgggacctcgccatcccagcccttgctgtctgactgctgccacttctgcactctgtctcgcagtctcctgaacccctctctccatgtgcctgctctctccctgcctcactggcccccatgttcccgaggtcccgcacccccaagcttcctccgtgcaggtctggtgtttccctgttttaagcacgtggacgcatacacagcacactcacacatgctccccttgcccgtgcctggctcctggctccaaggcctggggaacagagccccattgagacaccctgacgtcaccccctcttcattctcccccccccaaagccgggaacaggccttcatgtatgctgtgtccgatgaggaaagccgccgttgtggtgggcttgggagtggggaagggtccctgccagaagtgggagccagggtccaggccctggaggaggggttgctcagggaaggggtttaacaagggggcctgggacagctcccagaatgcggcttgaggcctttccagagcccccccacctcctttccgagttccctttgtgttgcatgtagtctctctcactccctctcttctgcaaagtttatttttagcctcctgctcccccgctcccaggactgtcgctgcacacacgttgcctggttaccgggacacggaggctggggcgggtgggctgggaagggtggctttaattttggggggagggtggaagtcagtacatcccccacccccagattgatgtctcaggggctttggccccccttctaggtctagagccagattggggctgaggtggaagtaacatggagcttggttccctgagaggggctgggagctttttggaggggatatgagggtccatgaagctggtggctgggtcacctgggttccaaaggaccctgggccttgcaggagaccttgtgatctggattctctagaagggtgaggataatgggttgaacacctagattcccaagagagcgatggtgctctggggtctctggaaggggttgtcaaaggctgtcacttttaaatggaacaggggctgttggtgctggtctgtgaacagctggaggtcatatccctggcaccctgaggggctggtggaaggctgggatatatctgaatggggcagggactccgggggctatgtggtactgcagcccagtctgggcaggagttgggggcctacagtgagctctggatttggagggggtggtccctgtgggagcaggaactactgattccctggattcccaaggggaaggatcccttgttggattccagacttgggtggagtctttgagggtggatgtgttgcctggaggccttgttggtacctgtttgagctggggccaagggaggcggattgacccctgcagcctgactggatttgggagccgcccatctttagctggccaccttggccttctctctctagtggcaccagggaccctgctctgcctccccactccttcccttcctcccctttccctcctcccacccctccattaccggctgcctggtgctgaattattgatggcccctgttgttgggaaatgacaacaaccgcagtccccccaccccccaccccccaccccaggctgcccaccccctccagagaccagccagcccaccgccaacctctacccaaggttaatgctgggggtgggcaggcaggtgggcagggaaggagctgaagatgagaaggcagaagtgggaagagggtgacagctccagggtgcctgctcaagctccaggcctgggctgggccctccagccaggtggggcctggatgcctgggtctggggagggcagtgtagctcagagggtccagggcttggggggcctctcagacagtctgggtttcactctccagggaagacagaagcctctgtgtgtgcaagcgctgacctcatcggcaaccgagtcaccccacccctcctggtccttttaagaaaaaaaaaaaaaatcttcctctcaggtcttggggttgggagtaggggctctgttgggtcttcggaggagacagtgcatgtttctaaggaggacatccgaggagctggggtggtgcaactggctctgaatgggagtgggggtaagctggggggctgtaccccaacccggctggccacgtgtgccccttcctgcccctcccccactggcagccctgcccgccagcctggcttggtgcctggccccctggcactggccccgggacccaccgccgacggtttcctgtttctcccggtgtcgctggagcgggctccagcttcccctcccccccagctcctgtctagcccctcacatgcagacagacagacacgcactgtcttctcttaaatcagatcttgacttggctgaggtgagacagatttccccatgttgaaggcagggcagaggtggaccctgggatgggacccagagccctggacttggcagcctgaccccaggccactccctgcctcccgtccccagggtttgccttcccggattgggcctacaagccagagtcgtcccctggctcaaggcagatccagctgtggcactttatcctggagctgctgcggaaggaggagtaccagggcgtcattgcctggcagggggactacggggaattcgtcatcaaagaccctgatgaggtggcccggctgtggggcgttcgcaagtgcaagccccagatgaattacgacaagctgagccgggccctgcggtgaggacgggctggggacccctgagcacatgtggagtgcccccccggggttgcacagccccttgcctgtgggaacaagcattgggtctgacccctggctttgccaaatcctagctgtgtgaccttgggccactcccagcccttccctgagccccttggtaacacagacgtatcctgaggagagattacagtcaccagtggatcctctagtcctgccttagatgtgaggagagagctggtacccaccctctgggcacttgatttgtcttttgtggcctacgttcctcagttggcctttgggaaccacacctaagtgcgtgttaaggtgtggagtctagacctgggtcccacgtacctgaccttcccaatggcatttctgtacccacagctattactataacaagcgcattctgcacaagaccaaggggaaacggttcacctacaagttcaatttcaacaaactggtgctggtcaattacccattcattgatgtggggttggctggtgagtaccagggatggttggtgtggagagggtctacatgccccttcaggctaggccaggggccttgacactgtctcccttctgccagggggtgcagtgccccagagtgccccgccagtgccgtcgggtggtagccacttccgcttccctccctcaacgccctccgaggtgctgtcccccaccgaggacccccgctcaccaccagcctgctcttcatcttcatcttccctcttctcggctgtggtggcccgccgcctgggccgaggctcagtcagtgactgtagtgatggcacgtcagagctggaggaaccgctgggagaggatccccgcgcccgaccacccggccctccggatctgggtgccttccgagggcccccgctggcccgcctgccccatgaccctggtgtcttccgagtctatccccggcctcggggtggccctgaacccctcagccccttccctgtgtcgcctctggccggtcctggatccctgctgccccctcagctctccccggctctgcccatgacgcccacccacctggcctacactccctcgcccacgctgagcccgatgtaccccagtggtggcggggggcccagcggctcagggggaggctcccacttctccttcagccctgaggacatgaaacggtacctgcaggcccacacccaaagcgtctacaactaccacctcagcccccgcgccttcctgcactaccctgggctggtggtgccccagccccagcgccctgacaagtgcccgctgccgcccatggcacccgagaccccaccggtcccctcctcggcctcgtcatcctcttcttcttcttcctccccattcaagtttaagctccagccgcccccactcggacgccggcagcgggcagctggggagaaggccgtagccggtgctgacaagagcggtggcagtgcaggcgggctggctgagggggcaggggcgctagccccaccgcccccgccaccacagatcaaggtggagcccatctcggaaggcgagtcggaggaggtagaggtgactgacatcagtgatgaggatgaggaagacggggaggtgttcaagacgccccgtgccccacctgcaccccctaagcctgagcccggcgaggcacccggggcatcccagtgcatgcccctcaagctacgctttaagcggcgctggagtgaagactgtcgcctcgaagggggtgggggccccgctgggggctttgaggatgagggtgaggacaagaaggtgcgtggggaggggcctggggaggctggggggcccctcaccccaaggcgggtgagctctgacctccagcatgccacggcccagctctccctggagcaccgagactcctgagggctgtgggcaggggacctgtgtgccccgcaccccccatgcttcttttgctgccttaagccccctatgccctggaggtgagggcagctctcttgtctcttccctgcctcctcccttttccctccccacattttgtataaaactttaatttcttttttttaaaaatggtgggggtgggtgggtgcccagggctaggggctattccctgtctctgtgggtttctaagctctgggcaaagtggtggtagggggagggagggggaagttaagggggtcacctccattctggggaatttatatttgaattgaggctttggccttaacacccaggaacttttctattacaatcgcttaggaagtaaagccttgtctccctccctgttctctgcctcttgtacccctctgacccacccgctctgccccactcccagccctcctcagccccagccctgcctgccctgcccctccagggggccatgagtgcctaggtttctcataccccacaaggtcacagcaggggagggagggacaattttataatgaaccaaaaattccatgtgttggggggtggggggcggaggagggtgaggggtgccgcccatgggccacaaatctctacaagtgcctgctatccctctcccactccccaccccagcaccggtccaaccccttcatccccagctgctcctaggactggcccatgggcaggcgggtggggggatgggaagggggtgccctgaaaccaaactggaagccccctctgcctcccagctggggcctctggggtggggtggggggctgtggtcaagccttattctgtattggggactgagggtggggggagtagaggggccgctggagaatgtattcaaaacaataaaactttggacctttggatgcagtgatcaatgtcctaggtgctcggtgcagccccctgagggccacagcttttgcttgattctgaaggaggggccccaccctggctgctagtgggtctgtggccagatcagtatagagggcctaaccctaatcagaatgggccaaatccaggcctgggaaaccgtctcctgacctcagaggcacatgagccccagagcttgatgatagtggtctcaggcatgcgccggcttcatcagactgctcctctgttccatggggaccttgtccttgaaagctcttcccactgtttttcaccacaaccccactgcccttgctcttctgattgcctggatgccttcccacttgtggctaggaattcttcccacaccccaggtccccagtgaagaccaggagctcccttttgcactgcccctcgcaccctgcccacacccctctcttgttgctctgagttcagctgggagtcctttggagaacagcctgtggtgtctcttgaaatgtagaccttgcaaggtaacttggtgatcttcaggtccaggtatgggaacacccccagccagcacacagccaagggactggctcactagtcatcgatgctgttgacctactgtgtgtccaatacttaagagctgcagtgttgtagctccttcagtatccagtaaggtaggggcaaccagtgctgctaagtgacttgcccagggccctgtggctgagccaggacttcaaagcccacgttctttcaacaactcctgacttttccttcctaacaccaagatagttggatccagatcaggctttcaggatcagaggatttgagatgagctcatagagctagaagctgagattggagagcttgaatcagggatgaaagaattaggaggaataatccaggctggaagtacccttagagacaagttctgtctccaccatgcaggtgaggtgcctgaggcccagaaaggggaaggggtttccccaaggccacacagctgctaagagatggagtg

SEQ ID NO: 9 - DNA, 21 bp, artificial sequences
  source 1..21: /mol_type=“DNA” /note=“Primer StopF2” /organism=“artificial sequences”
accgagattcctgagagctat

SEQ ID NO: 10 - DNA, 21 bp, artificial sequences
  source 1..21: /mol_type=“DNA” /note=“Primer 5578R” /organism=“artificial sequences”
agagactaaagagagctgtcc

SEQ ID NO: 11 - DNA, 21 bp, artificial sequences
  source 1..21: /mol_type=“DNA” /note=“Primer Intrl-2” /organism=“artificial sequences”
atcatacatgtttctgagggg

SEQ ID NO: 12 - DNA, 28 bp, artificial sequences
  source 1..28: /mol_type=“DNA” /note=“Primer mErf F2” /organism=“artificial sequences”
gctggagagaaggctctaggaggcactg

SEQ ID NO: 13 - DNA, 29 bp, artificial sequences
  source 1..29: /mol_type=“DNA” /note=“Primer mErf RI” /organism=“artificial sequences”
ggttaaggcagcaaaagctcagggagtgg